(19) United States
(12) Patent Application Publication
(10) Pub. No.: US 2024/0041396 A1
(43) Pub. Date: Feb. 8, 2024

(54) METHOD AND DEVICE FOR MONITORING SLEEP STAGE USING SLEEP PREDICTION MODEL

(71) Applicant: MELLOWING FACTORY CO., LTD., Seoul (KR)

(72) Inventor: Joonyong Lee, Seoul (KR)

(21) Appl. No.: 18/187,759

(22) Filed: Mar. 22, 2023

(30) Foreign Application Priority Data
Aug. 8, 2022 (KR) 10-2022-0098659
Dec. 28, 2022 (KR) 10-2022-0186923

(51) Int. Cl. A61B 5/00; A61B 5/11

(52) U.S. Cl. CPC A61B 5/4812; A61B 5/7264; A61B 5/1102; A61B 5/4818; A61B 2560/0238

(57) ABSTRACT

A health condition monitoring device according to an embodiment of the present application includes a memory and at least one processor. The at least one processor is configured to obtain a first bio-signal and a second bio-signal measured during sleep of the user; generate an expected sleep stage using a first neural network model based on the first bio-signal; detect a non-sleep stage of the user based on the second bio-signal; and generate a corrected sleep stage based on the result of detecting the non-sleep stage.

### CROSS-REFERENCE TO RELATED PATENT APPLICATION

This application claims the benefit of Korean Patent Application No. 10-2022-0098659, filed on Aug. 8, 2022, and Korean Patent Application No. 10-2022-0186923, filed on Dec. 28, 2022, both in the Korean Intellectual Property Office, the disclosures of which are incorporated herein in its entirety by reference.

### BACKGROUND OF THE INVENTION

### 1. Field of the Invention

The present invention relates to a method for extracting an indicator through an analysis of a bio-signal generated during sleep and monitoring a health condition based on the indicator, and a device for performing the same task.

### 2. Description of the Related Art

Since the quality of sleep is important that it affects the quality of life, the importance of sleep is being emphasized in the modern society. Recently, sleep-tech, or technology-based services that can help sleep in various ways, are attracting attention.

In this regard, an accurate technology for effectively measuring and analyzing bio-signal are essential in order to improve the quality of sleep by analyzing bio-signals generated from the human body during sleep and monitoring sleep stages, or predicting diseases or illnesses in advance by monitoring health.

However, in the past, in order to measure bio-signals related to sleep and health, the user had to sleep while wearing an electronic device on the body, and thus there was a limit in user convenience. The bio-signal acquired during sleep are irregular, and thus there was a limit to implementing an algorithmic model for accurately analyzing the bio-signal.

Accordingly, there is a demand for the development of a device capable of accurately measuring the bio-signal even in a state where the user does not directly wear the device on the body, the implementation of the model for accurately analyzing the measured bio-signal, and the development of a method for effectively delivering the result of monitoring the sleep stage or the health condition to the user.

### SUMMARY OF THE INVENTION

The present invention provides a method for extracting an indicator from a bio-signal generated during sleep and monitoring health, and a device and system for performing the same task.

The problems to be solved by the present invention are not limited to those described above, and problems not mentioned will be clearly understood by those of ordinary skill in the art from this specification and the accompanying drawings.

A health condition monitoring device according to an embodiment of the present application includes a memory and at least one processor. The at least one processor acquires a first bio-signal from a first device, extracts a first indicator and a second indicator based on an analysis of the first bio-signal, and monitors a health condition based on the first indicator and the second indicator. The first device is a device for acquiring the bio-signal generated from a first body part, the first body part is a part corresponding to the upper body, and the first bio-signal is a ballistocardiogram signal.

The solutions to the problems of the present invention are not limited to those described above, and solutions not mentioned will be clearly understood by those of ordinary skill in the art from this specification and the accompanying drawings.

According to embodiments of the present application, health condition monitoring with high reliability can be performed by measuring a bio-signal generated during sleep and analyzing the bio-signal using a pre-trained neural network model.

According to the embodiments of the present application, an indicator that is the basis for health condition monitoring can be accurately extracted by performing verification on whether or not the bio-signal generated during sleep is valid.

According to the embodiments of the present application, it is possible to provide a device capable of acquiring the bio-signal with high accuracy even when the user is not directly wearing the device in order to effectively measure the bio-signal generated during sleep.

According to the embodiments of the present application, the indicator that is the basis for health condition monitoring can be more accurately extracted by accurately analyzing complex and various types of ballistocardiogram signals acquired during sleep through a pre-trained neural network model.

According to the embodiments of the present application, a phase of sleep can be monitored based on the bio-signal generated during sleep, and a suitable time for the user to wake up can be determined through this, and an alarm can be provided to the user at the corresponding time point to induce the user to wake up.

Effects of the present invention are not limited to those described above, and effects not mentioned will be clearly understood by those of ordinary skill in the art from this specification and the accompanying drawings.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram for describing a method for acquiring a bio-signal and analyzing the bio-signal according to an embodiment;

FIGS. 2 and 3 are diagrams for describing a bio-signal analysis system according to an embodiment

FIG. 4 is a diagram for illustratively describing a method for extracting an indicator by measuring and acquiring the bio-signal by a bio-signal measuring device according to an embodiment;

FIGS. 5 to 7 are diagrams for illustratively describing types of bio-signals that can be acquired by the bio-signal measuring device, types of indicators that can be extracted from the bio-signals, and types of diseases that can be determined through an analysis of the indicators according to an embodiment;

FIG. 8 is a diagram for illustratively describing types of bio-signals acquired by a plurality of bio-signal measuring devices, types of indicators that can be extracted from the bio-signals, and types of diseases that can be determined through an analysis of the indicators according to an embodiment;

FIG. 9 is a diagram for illustratively describing a method for performing an analysis of apnea or blood pressure through the bio-signal measuring device according to an embodiment;

FIG. 10 is a diagram for illustratively describing a method for monitoring a sleep stage through the bio-signal measuring device according to an embodiment;

FIGS. 11 to 13 are diagrams for illustratively describing a method performed by the bio-signal measuring device to improve an accuracy of indicator extraction according to an embodiment;

FIGS. 14 and 15 are diagrams for describing a method for determining whether the placement of the bio-signal measuring device is appropriate according to an embodiment;

FIGS. 16 to 18 are diagrams for illustratively describing a method for acquiring probability values for heart rate and breathing rate in order to determine whether the placement of the bio-signal measuring device is appropriate according to an embodiment;

FIGS. 19 and 20 are diagrams for illustratively describing a system for acquiring a target bio-signal according to an embodiment;

FIG. 21 is a diagram for illustratively describing the bio-signal measuring device according to an embodiment;

FIG. 22 is a diagram for describing a configuration of the bio-signal measuring device according to an embodiment;

FIG. 23 is a diagram for illustratively describing a structure of a first bio-signal measuring device according to an embodiment;

FIG. 24 is a diagram for illustratively describing a structure of a first bio-signal measuring device according to another embodiment;

FIGS. 25 to 27 are diagrams for illustratively describing a method for monitoring a sleep stage based on a pressure value around the eye by the bio-signal measuring device according to an embodiment;

FIGS. 28 to 30 are diagrams for illustratively describing a method for determining the movement of the eye and a method for monitoring the sleep stage by the bio-signal measuring device according to an embodiment;

FIG. 31 is a diagram for illustratively describing a method for determining the movement of the eye and a method for monitoring the sleep stage by the bio-signal measuring device according to another embodiment;

FIGS. 32 to 34 are diagrams for illustratively describing a neural network model operable in the bio-signal measuring device according to an embodiment;

FIG. 35 is a diagram for describing a detailed method for monitoring the sleep stage using a pre-trained neural network model by the bio-signal measuring device according to an embodiment;

FIGS. 36 to 38 are diagrams for describing a method for correcting a predicted sleep stage using an additional indicator by the bio-signal measuring device according to an embodiment;

FIGS. 39 and 40 are diagrams for describing a method for correcting the predicted sleep stage using an additional indicator by the bio-signal measuring device according to another embodiment;

FIGS. 41 and 42 are diagrams for describing a method for providing an alarm to a user based on a sleep monitoring result by the bio-signal measuring device according to an embodiment;

FIG. 43 is a diagram for describing a method for providing the alarm to the user through a peripheral device based on the sleep monitoring result by the bio-signal measuring device according to an embodiment;

FIGS. 44 to 47 are diagrams for illustratively describing a method for a user terminal to guide the user on how to use the bio-signal measuring device according to an embodiment; and

FIGS. 48 to 52 are diagrams for illustratively describing a method for outputting a sleep stage monitoring result to the user by the user terminal according to an embodiment.

### DETAILED DESCRIPTION OF THE INVENTION

Hereinafter, preferred embodiments will be described in more detail with reference to the accompanying drawings. Moreover, detailed descriptions of well-known functions or configurations will be omitted in order not to unnecessarily obscure the focus of the present invention.

The above-described objects, features and, advantages of the present invention will be clearly understood through the following detailed description taken in conjunction with the accompanying drawings. However, while the present invention may have various modifications and alternative forms, specific embodiments thereof are shown by way of example in the accompanying drawings and will be described in detail herein.

Like reference numerals refer to like elements in principle throughout this specification. In addition, elements having the same function within the scope of the same idea shown in the drawings of each embodiment will be described using the same reference numerals, and overlapping descriptions thereof will be omitted.

When it is determined that detailed descriptions of related well-known functions or configurations may unnecessarily obscure the gist of the present invention, detailed descriptions thereof will be omitted. Further, the ordinal numbers (for example, first, second, etc.) used in description of the specification are used only to distinguish one element from another element.

Further, the term “module,” “unit,” “part,” or “component” of an element used herein is assigned or incorporated for convenience of specification description, and the term itself does not have a distinct meaning or role.

In the following embodiments, the singular forms “a” and “an” are intended to also include the plural forms, unless the context clearly indicates otherwise.

In the following embodiments, the terms “comprise,” “comprising,” “include,” and/or “including,” when used herein, specify the presence of stated features or elements described in the specification, but do not preclude the presence or addiction of one or more other features or elements.

In the drawing, the size of the elements may be exaggerated or reduced for convenience of description. For example, the size and thickness of each element shown in the drawings are arbitrarily indicated for the convenience of description and the present invention is not necessarily limited to the illustration.

When a certain embodiment is allowed to be implemented differently, a specific process order may be performed differently from the described order. For example, two consecutively described processes may be performed substantially at the same time or performed in an order opposite to the described order.

In the following embodiments, when elements and the like are described as being connected to each other, the elements are directly connected to each other or the elements are indirectly connected to each other with another element interposed therebetween.

For example, in this specification, when elements and the like are described as being electrically connected to each other, the elements are directly and electrically connected to each other or the elements are indirectly and electrically connected to each other with another element interposed therebetween.

1. Entire Process

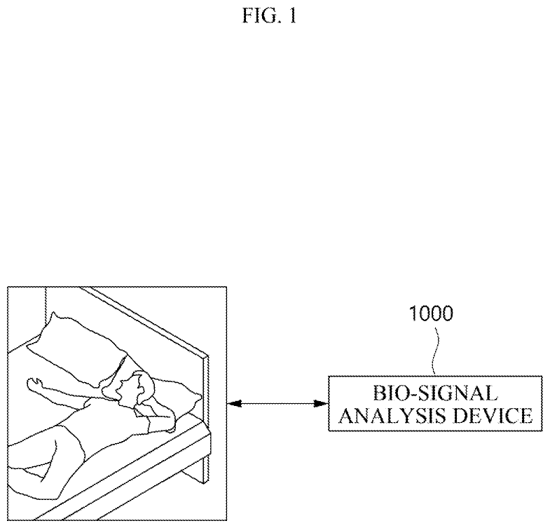

FIG. 1 is a diagram for describing a method for acquiring a bio-signal and analyzing the bio-signal according to an embodiment.

Referring to FIG. 1, the method for acquiring a bio-signal and analyzing the bio-signal according to an embodiment may be performed by a bio-signal analysis device 1000. The bio-signal analysis device 1000 includes at least one sensor, and may acquire at least one bio-signal from a body using the at least one sensor. The bio-signal analysis device 1000 may acquire at least one bio-signal from the body and then analyze the acquired bio-signal to perform an analysis related to a human health condition.

According to an embodiment, the bio-signal analysis device 1000 may acquire the bio-signal during sleep and analyze the bio-signal acquired during sleep to perform an analysis related to the human health condition. The bio-signal analysis device 1000 may monitor a sleep stage of a user by analyzing the bio-signals acquired during continued sleep, and may provide various feedbacks (e.g., providing sleep analysis results, suggesting plans to improve a sleeping environment, providing wake-up alarms at the optimal timing, etc.) based on this.

There are various bio-signals that can be acquired from the body during continued sleep. As will be described later, the bio-signal analysis device 1000 may acquire data such as a ballistocardiogram signal, various types of sounds, pressure around the eye, a photoplethysmogram (PPG) value, temperature, and humidity from the body during continued sleep. Since the various types of data described above can be continuously acquired during sleep, and accumulated data acquired over several days or months can be acquired in some cases, the health condition can be monitored with higher accuracy based on the cumulatively acquired data.

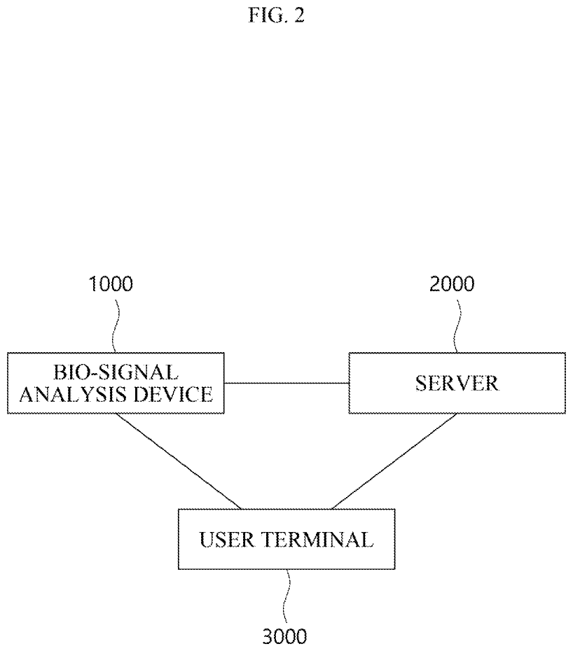

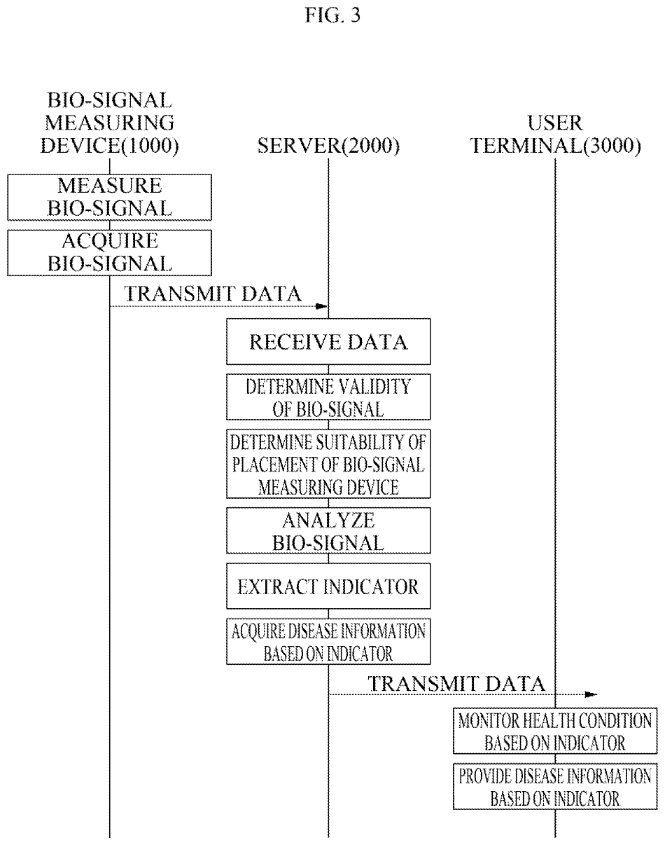

FIGS. 2 and 3 are diagrams for describing a bio-signal analysis system according to an embodiment. Referring to FIG. 2, the bio-signal analysis system may include a bio-signal measuring device 1000, a server 2000, and a user terminal 3000.

According to an embodiment, the bio-signal measuring device 1000 may acquire at least one or more various types of bio-signals from the user in a contact or non-contact manner. The at least one bio-signal acquired from the bio-signal measuring device 1000 may be transmitted to the server 2000, and the server 2000 may analyze the received at least one bio-signal to monitor the health condition of the user.

A result of the health condition monitoring of the user performed by the server 2000 may be transmitted to the bio-signal measuring device 1000 or the user terminal 3000, and the bio-signal measuring device 1000 or the user terminal 3000 may provide feedback to the user based on the health condition monitoring result. Meanwhile, the server 2000 may perform training of a neural network model for extracting the indicator with which the sleep stage can be monitored based on the bio-signal.

More specifically, referring to FIG. 3, the bio-signal measuring device 1000 may measure the bio-signal from the user, acquire and store the measured bio-signal. The bio-signal measuring device 1000 may transmit the acquired bio-signal to the server 2000. A method for measuring the bio-signal by the bio-signal measuring device 1000 and a detailed description of a hardware structure of the device related thereto will be described later.

The server 2000 may determine the validity of the bio-signal based on the received data, and when it is determined that the bio-signal is valid, the server 2000 may determine whether the placement of the bio-signal measuring device 1000 is appropriate. A specific method for the server 2000 to determine the validity of the bio-signal and to determine whether the placement of the bio-signal measuring device 1000 is appropriate will be described later.

The server 2000 may output the indicators by analyzing the acquired bio-signal when it is determined that the bio-signal data is valid and the placement of the bio-signal measuring device 1000 is appropriate. In this case, the server 2000 may extract the indicator from the acquired bio-signal using a pre-trained neural network model. A detailed method for extracting the indicators from the bio-signal and the types of indicators acquired from the bio-signal will be described later.

The server 2000 may perform monitoring on disease information or sleep stage through an analysis of the extracted indicator. Thereafter, the server 2000 may transmit the extracted indicator or the monitoring result of the sleep stage to the user terminal 3000, and the user terminal 3000 may provide feedback to the user based on the received data. A detailed description of a method in which the server 2000 acquires disease information or perform monitoring on the sleep stage and a method in which the user terminal 3000 provides feedback to the user will be described later.

Meanwhile, although not illustrated in the drawing, according to another embodiment, the bio-signal measuring device 1000 may acquire at least one or more bio-signals of various types from the user in a contact or non-contact manner, and analyze the acquired at least one bio-signal to monitor the health condition of the user. In this case, the server 2000 may perform training of the neural network model for extracting the indicator with which the sleep stage can be monitored based on the bio-signal, and the bio-signal measuring device 1000, after receiving information about the trained neural network model from the server 2000, may analyze at least one bio-signal described above using the received information.

The bio-signal analysis device 1000 may provide feedback to the user based on the health condition monitoring result acquired by analyzing at least one bio-signal. The bio-signal analysis device 1000 may transmit the health condition monitoring result to the user terminal 3000, and the user terminal 3000 may provide feedback to the user based on the health condition monitoring result.

Hereinafter, for convenience of description, it is described that an operation of measuring and acquiring the bio-signal from the body and analyzing the acquired bio-signal is performed by the bio-signal measuring device 1000, but is not limited thereto, and the operation described above and operations corresponding thereto may also be performed by the server 2000 or the user terminal 3000.

2. Extraction of Indicators from Body Signals

As described above, there are various bio-signals that can be measured and acquired during sleep, and various types of health-related monitoring can be performed by analyzing the bio-signals. In order to acquire highly accurate monitoring results, it is important to specify a bio-signal related to the health condition to be monitored and accurately measure and acquire the bio-signal, and at the same time, it is important to accurately analyze the measured and acquired bio-signal to extract indicators.

The bio-signal measuring device 1000 according to an embodiment may acquire at least one bio-signal measured during sleep and extract the indicator through an analysis of the acquired bio-signal.

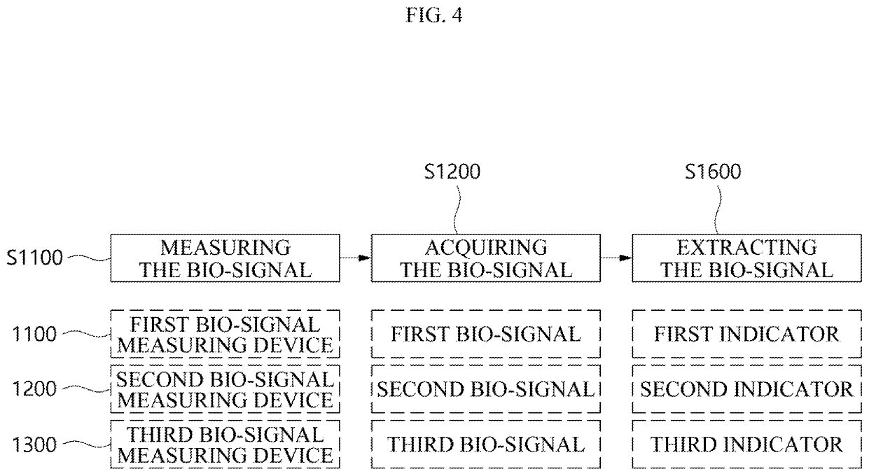

FIG. 4 is a diagram for illustratively describing a method for extracting the indicator by measuring and acquiring the bio-signal by the bio-signal measuring device according to an embodiment.

Referring to FIG. 4, the bio-signal measuring device 1000 according to an embodiment may perform steps of measuring the bio-signal (S1100), acquiring the bio-signal (S1200), and extracting the indicator (S1600).

The bio-signal measuring device 1000 may measure the bio-signal using at least one sensor. The bio-signal measuring device 1000 may include a first bio-signal measuring device 1100, a second bio-signal measuring device 1200 and a third bio-signal measuring device 1300. The same or different sensors may be provided in the first bio-signal measuring device 1100 to the third bio-signal measuring device 1300, and different types of bio-signals may be measured depending on a place or location where the devices are placed.

For example, at least one force sensor may be provided in the first bio-signal measuring device 1100 and may be placed to measure the bio-signal generated from an upper part of the human body (e.g., waist, chest, back, arm parts of the human body). In this case, the first bio-signal measuring device 1100 may be provided to measure a first bio-signal generated from the upper body part of the human body (e.g., waist, chest, back, arm parts of the human body) using at least one force sensor. In addition, at least one force sensor may be provided in the first bio-signal measuring device 1100 and may be placed to measure the bio-signal generated from a lower part of the human body (e.g., waist, buttocks, thighs, calves, feet of the human body). In this case, the first bio-signal measuring device 1100 may be provided to measure a first bio-signal generated from the lower part of the human body (e.g., waist, buttocks, thighs, calves, feet of the human body) using at least one force sensor.

As another example, at least one force sensor and at least one sound sensor may be provided in the second bio-signal measuring device 1200, and may be placed to measure the bio-signal generated from the head of the human body. In this case, the second bio-signal measuring device 1200 may be provided to measure the first bio-signal using at least one force sensor, and may be provided to measure a second bio-signal using at least one sound sensor.

However, this is just an example, and various sensors may be provided in the first bio-signal measuring device 1100 to the third bio-signal measuring device 1300, and may be provided to measure various bio-signals according to a placement place or a placement method thereof. The structural characteristics of the bio-signal measuring device 1000 will be described later.

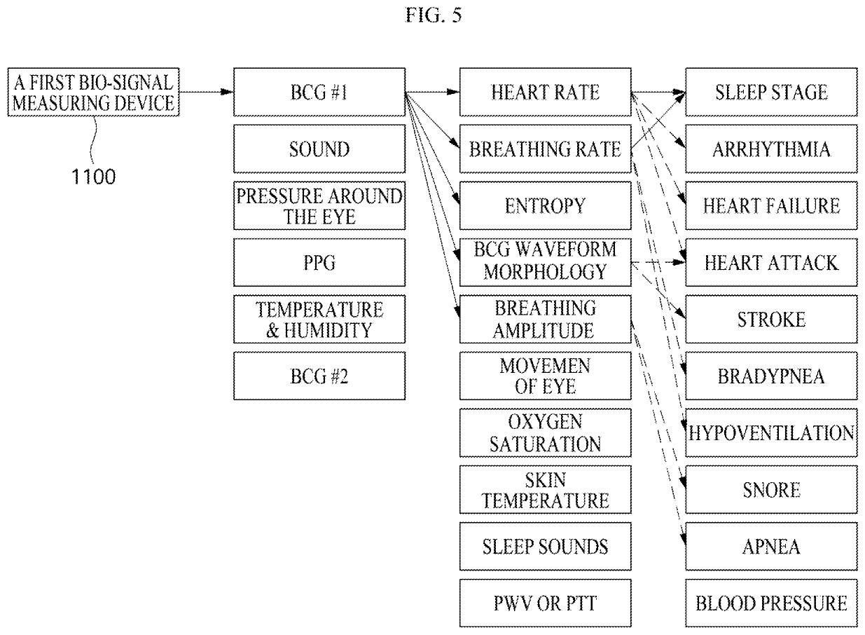

FIGS. 5 to 7 are diagrams for illustratively describing the types of bio-signals that can be acquired by the bio-signal measuring device, types of indicators that can be extracted from the bio-signals, and types of diseases that can be determined through an analysis of the indicators according to an embodiment.

Referring to FIGS. 5 to 7, the bio-signal measuring device 1000 may measure and acquire various bio-signals. For example, the bio-signal measuring device 1000 may acquire data related to at least one of the ballistocardiogram (BCG) signal, sound, pressure around the eye, a photoplethysmogram (PPG) value, temperature, and humidity using at least one sensor.

The bio-signal measuring device 1000 may extract various types of indicators based on data related to the acquired bio-signals. For example, the bio-signal measuring device 1000 may extract the indicator related to at least one of heart rate, breathing rate, entropy, BCG waveform morphology, breathing amplitude, movement of the eye, oxygen saturation, skin temperature, sleep sounds, pulse wave velocity (PWV), and pulse transit time (PTT) based on data related to the bio-signal.

The bio-signal measuring device 1000 may perform an analysis of a disease or a specific indicator using the data related to the acquired bio-signal and at least one of the extracted indicators. For example, the bio-signal measuring device may perform an analysis of at least one of sleep stage, arrhythmia, heart failure, heart attack, stroke, bradypnea, hypoventilation, snore, apnea, and blood pressure 1000 using the data related to the acquired bio-signal and at least one of the extracted indicators.

As described above, depending on the type, placement place, and placement method of the bio-signal measuring device 1000, the types of bio-signals that can be acquired by the bio-signal measuring device 1000, the types of indicators to be extracted, and the types of diseases that can be analyzed may be different.

As an example, referring to FIG. 5, the first bio-signal measuring device 1000 may measure and acquire the ballistocardiogram signal generated from the upper body part of the human body, and may extract at least one of heart rate, breathing rate, entropy, BCG waveform morphology, and breathing amplitude from the ballistocardiogram signal using a pre-trained neural network model.

In this case, the ballistocardiogram signal relates to a signal that the body vibrates with the heartbeat, and may mean the momentum of the blood pumped by the heart. In addition, since the ballistocardiogram signal is based on the vibrations of the upper body, information about breathing can also be measured when measuring the ballistocardiogram signal. The ballistocardiogram signal described in this present application may be defined as a pulse signal or a pulse signal based on vibration. The ballistocardiogram signal may be a signal (e.g., the pulse signal or the pulse signal based on vibration) measured at at least one of all parts (e.g., all parts of the body including the head, upper body, and lower body) of the human body. Hereinafter, for convenience of description, the term ballistocardiogram signal is used, but this may also be defined as the pulse signal or the pulse signal based on vibration.

The first bio-signal measuring device 1000 may perform analysis on at least one of the arrhythmia, heart failure, and heart attack using a pre-trained neural network model from the extracted heart rate. The first bio-signal measuring device 1000 may perform analysis on at least one of the bradypnea and hypoventilation using a pre-trained neural network model from the extracted breathing rate. The first bio-signal measuring device 1000 may perform analysis of the sleep stage using a pre-trained neural network model from the extracted heart rate and breathing rate. The first bio-signal measuring device 1000 may perform analysis of the stroke using a pre-trained neural network model from the extracted BCG waveform morphology. The first bio-signal measuring device 1000 may perform analysis of the heart attack using a pre-trained neural network model from the extracted heart rate and BCG waveform morphology. The first bio-signal measuring device 1000 may perform analysis on at least one of the snore and apnea using a pre-trained neural network model from the extracted breathing amplitude. However, this is illustrative, and the first bio-signal measuring device 1000 may extract various types of the indicators from the acquired ballistocardiogram signal and perform analysis on various types of diseases based on the extracted indicators.

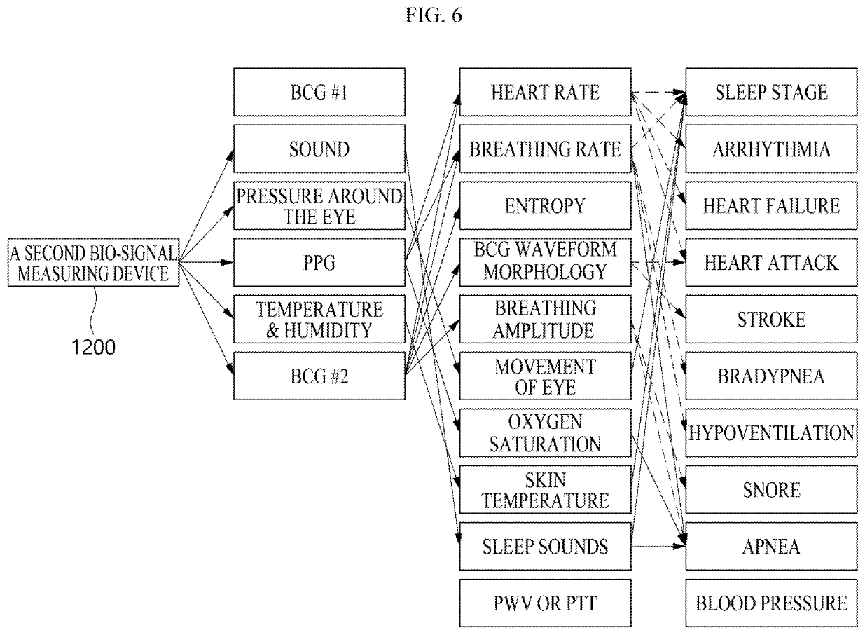

As another example, referring to FIG. 6, the second bio-signal measuring device 1200 may measure various types of bio-signals generated from the head of the human body. For example, the second bio-signal measuring device 1200 may acquire at least one of the sound, pressure around the eye, PPG value, temperature, humidity, and ballistocardiogram signal.

The second bio-signal measuring device 1200 may extract data about sleep sounds from the indicator related to sound. The second bio-signal measuring device 1200 may extract data about the movement of the eye from a pressure value around the eye. The second bio-signal measuring device 1200 may extract data about at least one of heart rate, breathing rate, and oxygen saturation from the PPG value. The second bio-signal measuring device 1200 may extract data about skin temperature from temperature and humidity values. The second bio-signal measuring device 1200 may extract at least one of heart rate, breathing rate, entropy, BCG waveform morphology, and breathing amplitude from the ballistocardiogram signal using a pre-trained neural network model.

The second bio-signal measuring device 1200 may perform analysis of sleep stage based on data about the extracted movement of the eye, skin temperature, and sleep sounds. The second bio-signal measuring device 1200 may perform the analysis of the sleep stage by additionally considering heart rate and breathing rate. For example, the second bio-signal measuring device 1200 analyzes and monitors the sleep stage based on heart rate and breathing rate, but may use at least one of the movement of the eye, sleep sounds, and skin temperature as an additional indicator of more accurate analysis of sleep stage.

The second bio-signal measuring device 1200 may perform analysis and diagnosis of apnea based on the indicators extracted from the bio-signal. The second bio-signal measuring device 1200 may perform analysis and diagnosis of apnea based on at least one of breathing rate, breathing amplitude, oxygen saturation, and sleep sounds.

The second bio-signal measuring device 1200 may also perform an analysis of heart rate, breathing rate, BCG waveform morphology and breath amplitude, which is the same as or corresponds to the analysis performed through the first bio-signal measuring device 1100, and thus duplicate description thereof will be omitted.

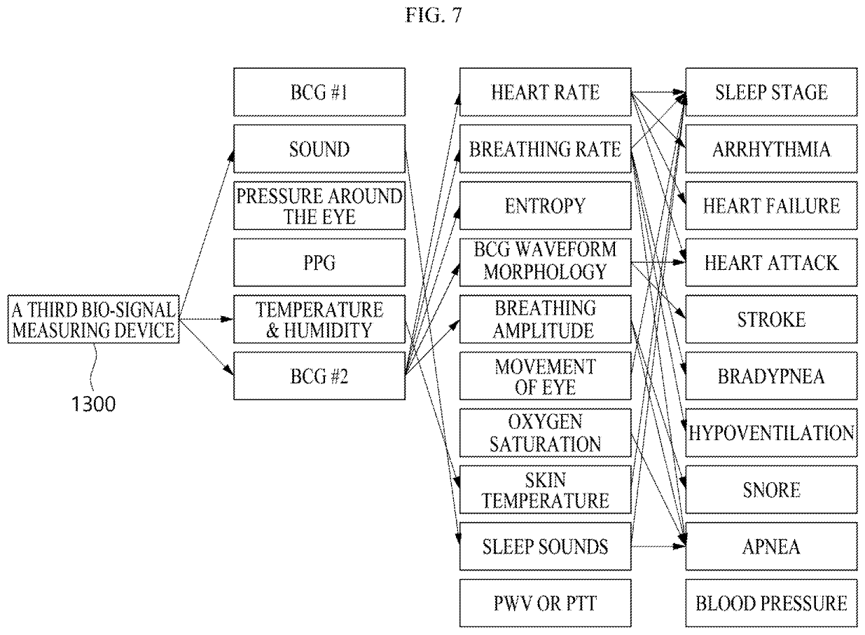

As another example, referring to FIG. 7, the third bio-signal measuring device 1300 may acquire data about at least one of the sound, temperature, humidity, and ballistocardiogram signal. The third bio-signal measuring device 1300 may extract the indicators of at least one of heart rate, breathing rate, entropy, BCG waveform morphology, breathing amplitude, skin temperature, and sleep sounds, and analyze diseases such as the sleep stage, arrhythmia, and heart failure using the extracted indicators. Since the operations described above performed by the third bio-signal measuring device 1300 are the same as or correspond to the operations described above with reference to FIGS. 5 and 6, duplicate description thereof will be omitted.

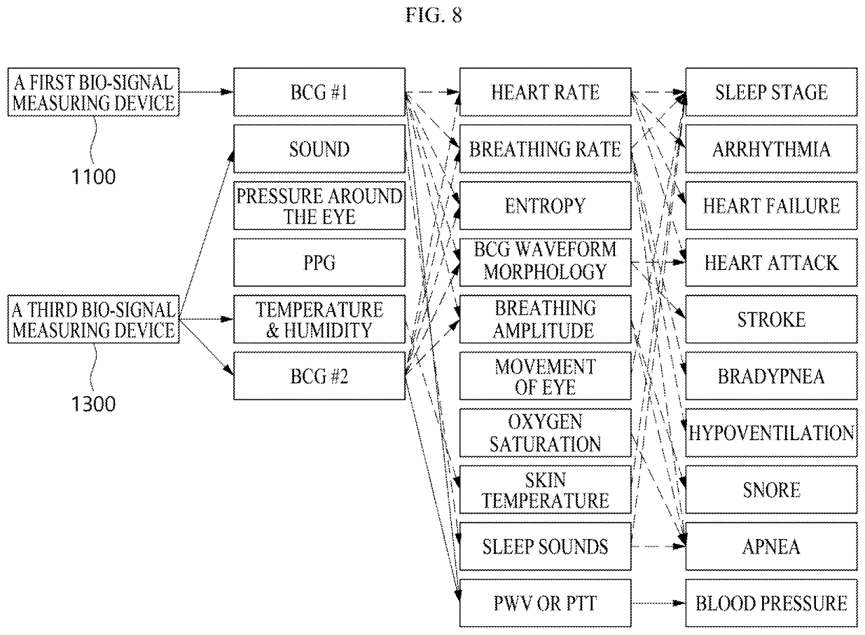

FIG. 8 is a diagram for illustratively describing the types of bio-signals acquired by a plurality of bio-signal measuring devices, types of indicators that can be extracted from the bio-signals, and types of diseases that can be determined through an analysis of the indicators according an embodiment.

Referring to FIG. 8, when the plurality of bio-signal measuring devices are placed, more various bio-signals can be measured from the human body, and specific types of indicators can be extracted using the measured bio-signals.

For example, the first bio-signal measuring device 1100 may be placed to measure a first part of the human body, and the third bio-signal measuring device 1300 may be placed to measure a second part of the human body. In this case, the first bio-signal measuring device 1100 acquires a first ballistocardiogram signal measured from the first part of the human body, and the third bio-signal measuring device 1300 may acquire a second ballistocardiogram signal measured from the second part of the human body. The first bio-signal measuring device 1100 or the third bio-signal measuring device 1300 may extract the PWV value or the PTT value based on the first ballistocardiogram signal and the second ballistocardiogram signal, and analyze the blood pressure based on the PWV value or PTT value.

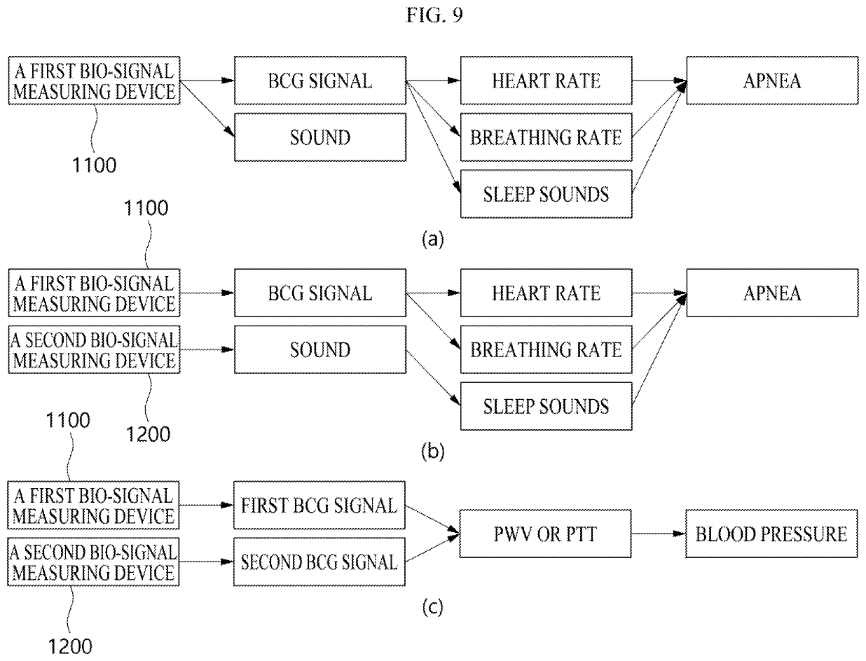

FIG. 9 is a diagram for illustratively describing a method for performing an analysis of apnea or blood pressure through the bio-signal measuring device according to an embodiment. Referring to FIG. 9, the bio-signal measuring device 1000 according to an embodiment may perform an analysis of apnea or blood pressure based on the acquired bio-signals.

Referring to (a) of FIG. 9, at least one force sensor and at least one sound sensor may be provided in the first bio-signal measuring device 1100. The first bio-signal measuring device 1100 may acquire the ballistocardiogram signal using the at least one force sensor, and may acquire data about sound using the at least one sound sensor.

The first bio-signal measuring device 1100 may extract a plurality of indicators from the ballistocardiogram signals using a pre-trained neural network model. The first bio-signals measuring device 1100 may extract a first indicator and a second indicator from the ballistocardiogram signals using a pre-trained neural network model. The first bio-signal measuring device 1100 may extract the indicators of breathing rate and breathing amplitude from the ballistocardiogram signal using the a-trained neural network model.

The first bio-signal measuring device 1100 may extract a third indicator from the data about sound using a pre-trained neural network model. The first bio-signal measuring device 1100 may extract the indicator related to sleep sounds from the data about sound using a pre-trained neural network model.

The first bio-signal measuring device 1100 may perform analysis or diagnosis of apnea based on the first indicator to the third indicator using a pre-trained neural network model. The first bio-signal measuring device 1100 may perform analysis or diagnosis of apnea based on the indicators of breathing rate, breathing amplitude, and sleep sounds using a pre-trained neural network model.

Illustratively, the first bio-signal measuring device 1100 may acquire the ballistocardiogram signal during a first time period, and extract the first indicator and the second indicator based on the ballistocardiogram signal using a pre-trained neural network model. The first bio-signal measuring device 1100 may determine apnea or sleep stage using at least one of the first indicator and the second indicator. The first bio-signal measuring device 1100 may acquire data about sound using the sound sensor during the first time period, and extract the third indicator related to sleep sounds based on the data about sound. The first bio-signal measuring device 1100 may update the determination result of apnea or sleep stage by additionally considering the third indicator.

According to one embodiment, the analysis or diagnosis of apnea is performed using a pre-trained neural network model, and the neural network model may be trained to acquire data about the presence or absence, frequency, or degree of apnea symptoms based on training data including at least one of breathing rate, breathing amplitude, and sleep sounds.

The neural network model can be trained using training data including data about breathing rate, breathing amplitude, and sleep sounds and labeling data. The labeling data may include a first labeling value corresponding to the presence or absence of apnea symptoms, a second labeling value corresponding to the frequency of apnea, and a third labeling value corresponding to the degree of apnea.

More specifically, the neural network model may acquire an output value after receiving at least one of breathing rate, the amplitude, and the sleep sounds. Thereafter, the neural network model may be trained by updating the neural network model based on an error value calculated by considering the difference between the output value and the labeling data. The output value may include a first output value corresponding to the first labeling value, a second output value corresponding to the second labeling value, and a third output value corresponding to the third labeling value.

According to another embodiment, the neural network model may be trained to acquire data related to apnea based on at least one of breathing rate, the breathing amplitude, and the sleep sounds using two neural network models (e.g., a first neural network model and a second neural network model).

For example, the first neural network model may be trained to acquire breathing state information by receiving data about breathing rate or the breathing amplitude. The second neural network model may be trained to acquire data related to apnea by receiving the breathing state information and data about sleep sounds.

The first neural network model and the second neural network model may mean independent and separate neural network models, but are not limited thereto, and may be physically or logically separated from one neural network model. That is, a first portion of the neural network model may be trained to acquire the breathing state information from breathing rate or breathing amplitude, and a second portion may be trained to acquire the data related to apnea from the breathing state information and sleep sounds.

Referring to (b) of FIG. 9, the analysis or diagnosis of apnea may be performed through a plurality of bio-signal measuring devices. For example, at least one force sensor may be provided in the first bio-signal measuring device 1100, and the first bio-signal measuring device 1100 may measure a ballistocardiogram signal using the at least one force sensor. At least one sound sensor may be provided in the second bio-signal measuring device 1200, and the second bio-signal measuring device 1200 may acquire the data about sound using the at least one sound sensor.

More specifically, the first bio-signal measuring device 1100 may be provided to measure and acquire the ballistocardiogram signal generated from the upper body part of the human body. In order to effectively acquire the ballistocardiogram signal generated from the human body during sleep, the bio-signal measuring device needs to be placed in a region adjacent to the upper body of the human body. Accordingly, the first bio-signal measuring device 1100 may be placed in the region adjacent to the upper body of the human body to measure and acquire the ballistocardiogram signal generated from the upper body part of the human body.

At least one sound sensor may be provided in the second bio-signal measuring device 1200, and the second bio-signal measuring device 1200 may acquire the data about sound generated during sleep using the at least one sound sensor. In order to more effectively acquire the data about sound generated during sleep, the bio-signal measuring device needs to be placed in a region adjacent to the head of the human body. Accordingly, the second bio-signal measuring device 1200 may be placed in the region adjacent to the head of the human body, and measure and acquire a sound signal generated from the head of the human body.

In other words, the first bio-signal measuring device 1100 may be placed in the region adjacent to the first part of the human body (e.g., upper body part of the human body) to measure the first bio-signal (e.g., ballistocardiogram signal), and the second bio-signal measuring device 1200 may be placed in a region adjacent to the second part of the human body (e.g., the head of the human body) to measure the second bio-signal (e.g., sound signal). In this case, the first part and the second part may be different parts of the human body, and the first bio-signal and the second bio-signal may be different bio-signals.

The indicators of breathing rate and breathing amplitude may be extracted based on the ballistocardiogram signal acquired from the first bio-signal measuring device 1100, and the indicator of the sleep sounds may be extracted based on the sound signal acquired from the second bio-signal measuring device 1200. The first bio-signal measuring device 1100 or the second bio-signal measuring device 1200 may perform analysis or diagnosis of apnea based on the extracted breathing rate, breathing amplitude, and sleep sounds. The analysis or diagnosis of apnea may be performed by the first bio-signal measuring device 1100, the second bio-signal measuring device 1200, or the server 2000.

On the other hand, the analysis or diagnosis of apnea based on at least one of breathing rate, breathing amplitude, and sound signal may be performed using a pre-trained neural network model. In this case, since the structure or training method for the neural network model is the same as or corresponds to that described through (a) of FIG. 9, duplicate description thereof will be omitted.

Referring to (c) of FIG. 9, a plurality of bio-signal measuring devices may acquire the pulse wave velocity (PWV) value or the pulse transit time (PTT) value using the ballistocardiogram signals, and determine blood pressure based on the PWV value or the PTT value.

The first bio-signal measuring device 1100 may acquire the first ballistocardiogram signal, and the second bio-signal measuring device 1200 may acquire the second ballistocardiogram signal.

The first bio-signal measuring device 1100 may be placed in a region adjacent to the first part of the human body to acquire the first ballistocardiogram signal, and the second bio-signal measuring device 1200 may be placed in a region adjacent to the second part of the human body to acquire the second ballistocardiogram signal. In this case, the first part and the second part may be different parts of the human body. The first part and the second part are different parts of the human body, and may be parts separated by a preset distance or more. Illustratively, the first part may be the upper body part of the human body, and the second part may be the head of the human body. As another example, the first part may be the upper body part of the human body, and the second part may be the lower body part of the human body.

The first bio-signals measuring device 1100 may acquire the first ballistocardiogram signal measured during a first time period, and the second bio-signal measuring device 1200 may acquire the second ballistocardiogram signal measured during a second time period. At least a portion of the first time period and at least a portion of the second time period may overlap each other. The first time period and the second time period may be determined to be greater than or equal to a predetermined minimum time period (e.g., 0.1 second to 0.4 second).

The first bio-signal measuring device 1100 or the second bio-signal measuring device 1200 may acquire the PWV value or the PTT value based on the first ballistocardiogram signal and the second ballistocardiogram signal. Thereafter, the first bio-signal measuring device 1100 or the second bio-signal measuring device 1200 may acquire a blood pressure value based on the PWV value or the PTT value.

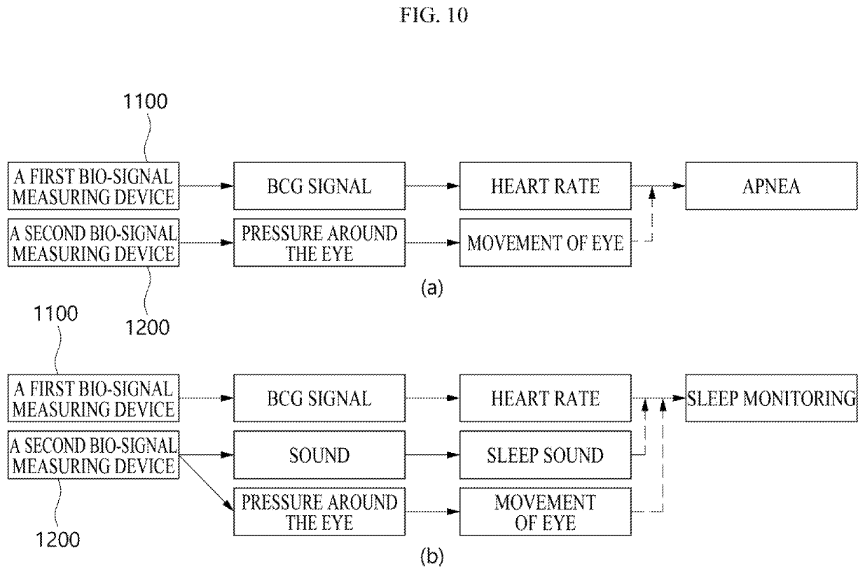

FIG. 10 is a diagram for illustratively describing a method for monitoring the sleep stage through the bio-signal measuring device according to an embodiment. Referring to FIG. 10, the bio-signal measuring device 1000 according to an embodiment may monitor the sleep stage based on at least one bio-signal.

Referring to (a) of FIG. 10, the first bio-signal measuring device 1100 may acquire the ballistocardiogram signal, and the second bio-signal measuring device 1200 may acquire the pressure value around the eye.

The first bio-signal measuring device 1100 may extract heart rate from the ballistocardiogram signal acquired using a pre-trained neural network model. Thereafter, the first bio-signal measuring device 1100 may monitor the sleep stage based on the extracted heart rate.

The first bio-signal measuring device 1100 monitors the sleep stage based on heart rate, but may utilize an additional indicator for more accurate monitoring. For example, the first bio-signal measuring device 1100 may use information about the movement of the eye as the additional indicator based on the pressure value around the eye measured by the second bio-signal measuring device 1200.

In other words, the first bio-signal measuring device 1100 monitors the sleep stage based on heart rate, but may additionally consider the movement of the eye to monitor the sleep stage. When the sleep stage is monitored by additionally considering the movement of the eye, it is possible to monitor the sleep stage with higher accuracy compared to monitoring the sleep stage only using heart rate.

Illustratively, the second bio-signal measuring device 1200 may acquire the pressure value around the eye, and extract information about the movement of the eye from the pressure value around the eye. The information about the movement of the eye may be related to left and right movement of the eye, up and down movement of the eye, degree of movement, magnitude of movement, frequency of movement, etc. The first bio-signal measuring device 1100 may monitor the sleep stage by utilizing the information about the movement of the eye described above as an additional indicator.

Meanwhile, according to another embodiment, the pressure value around the eye may be acquired by the second bio-signal measuring device 1200 or may be acquired from the first bio-signal measuring device 1100 from which the ballistocardiogram signal is acquired. For example, the first bio-signal measuring device 1100 may acquire the ballistocardiogram signal and the pressure value around the eye, and extract data about heart rate and the movement of the eye based thereon.

Referring to (b) of FIG. 10, the second bio-signals measuring device 1200 may additionally acquire a sound signal and extract an indicator related to sleep sounds based on the sound signal.

The first bio-signal measuring device 1100 monitors the sleep stage based on heart rate, but may monitor the sleep stage by additionally considering the indicator related to the sleep sounds. In addition, the first bio-signal measuring device 1100 monitors the sleep stage based on heart rate, but may monitor the sleep stage by additionally considering the indicator related to the sleep sounds and the information about the movement of the eye. When the sleep stage is monitored by additionally considering the indicator related to the sleep sound, the sleep stage can be monitored with higher accuracy compared to monitoring the sleep stage only using heart rate.

Although not illustrated in the drawing, the first bio-signal measuring device 1100 or the second bio-signal measuring device 1200 may provide an alarm to the user based on the sleep stage monitoring result. For example, the first bio-signal measuring device 1100 may provide a first alarm to the user based on the sleep stage monitoring result, and the second bio-signal measuring device 1200 may provide a second alarm to the user based on the sleep stage monitoring result.

Illustratively, the first bio-signal measuring device 1100 may be provided to measure and acquire the ballistocardiogram signal generated from then upper body part of the human body. In this case, the first bio-signal measuring device 1100 may provide a vibration alarm to the user based on the sleep stage monitoring result. Since the first bio-signal measuring device 1100 is placed in the region adjacent to the upper body part of the human body, the alarm can be effectively provided to the user through vibration.

As another example, the second bio-signal measuring device 1200 may be provided to acquire the sound signal generated from the head of the human body or the pressure value around the eye. In this case, the second bio-signal measuring device 1200 may provide a sound alarm or an alarm (e.g., LED alarm) providing a visual effect to the user based on the sleep stage monitoring result. Since the second bio-signal measuring device 1200 is placed in the region adjacent to the head of the human body, the alarm can be effectively provided to the user through sound or light.

A more specific method for monitoring the sleep stage based on the bio-signal acquired from the bio-signal measuring device and a more specific method for providing an alarm to the user based on the monitoring result will be described later with reference to the drawings.

3. Method for Improving Accuracy of Indicator Extraction

As described above, the bio-signal measuring device 1000 may monitor the health condition based on the indicator extracted from the bio-signal. In this case, for more accurate monitoring of the health condition, it is necessary to accurately extract the indicators that are the basis therefor.

For example, in order to accurately extract the indicator, the bio-signal should be measured in a correct way using the bio-signal measuring device 1000, and verification of whether the measured bio-signal is a valid signal that can be a target of analysis should be performed.

In particular, when the bio-signal is measured by directly or indirectly contacting the human body, a process of determining whether the measuring device is in correct contact with the human body is essentially required. In addition, if it is intended to acquire the bio-signal generated during sleep, it is necessary to determine whether the bio-signal being acquired is a signal measured during sleep or whether the bio-signal being acquired satisfies other predetermined conditions.

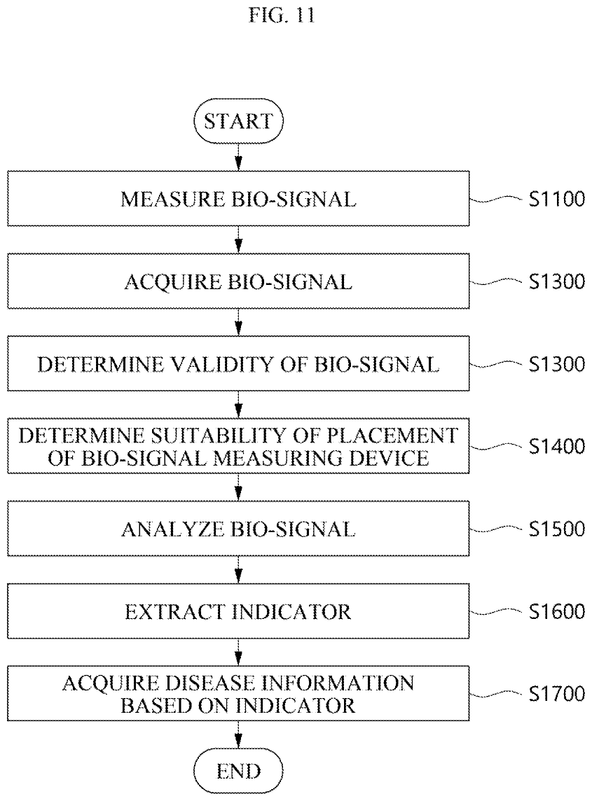

FIGS. 11 to 13 are diagrams for illustratively describing a method performed by the bio-signal measuring device to improve an accuracy of indicator extraction according to an embodiment.

Referring to FIG. 11, the bio-signal measuring device 1000 may perform a step of measuring the bio-signal (S1100), a step of acquiring the bio-signal (S1200), a step of determining the validity of the bio-signal (S1300), a step of determining the legitimacy of the placement of the bio-signal measuring device (S1400), a step of analyzing the bio-signal (S1500), a step of extracting the indicator (S1600), and a step of acquiring disease information based on the indicator (S1700).

As described above, the bio-signal measuring device 1000 may measure and acquire the bio-signals generated during sleep through the step of measuring the bio-signal (S1100), the step of acquiring the bio-signal (S1200), the step of analyzing the bio-signal (S1500), the step of extracting indicators (S1600), and the step of acquiring disease information based on the indicator (S1700), extract the indicator therefrom, and acquire the disease information based on the indicator.

The bio-signal measuring device 1000 may additionally perform the step of determining the validity of the bio-signal (S1300) and the step of determining the legitimacy of the placement of the bio-signal measuring device (S1400) in order to improve the accuracy of the extracted indicator.

The bio-signal measuring device 1000 may determine whether or not the measured bio-signal is valid through the step of determining the validity of the bio-signal (S1300). The step of determining the validity of the bio-signal (S1300) may include a first step of determining whether a user is located on the bio-signal measuring device 1000 using a first algorithm, and a second step of determining whether the measured bio-signal satisfies a predetermined criterion using a second algorithm.

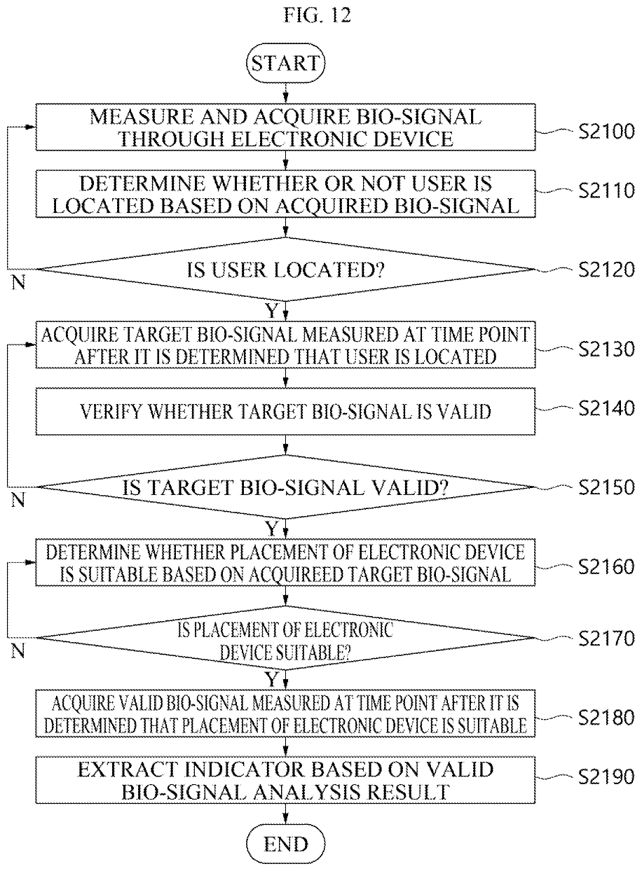

More specifically, referring to FIG. 12, the bio-signal measuring device 1000 may determine whether or not the user is located on the bio-signal measuring device 1000 based on an electronic signal input to the bio-signal measuring device 1000 using the first algorithm (S2110).

When it is determined that the user is located on the bio-signal measuring device 1000, the bio-signal measuring device 1000 may acquire a target bio-signal to be measured at a time point after it is determined that the user is located (S2130).

The bio-signal measuring device 1000 may verify whether the acquired target bio-signal is valid using the second algorithm (S2140). After acquiring the ballistocardiogram signal, the bio-signal measuring device 1000 may determine whether the ballistocardiogram signal is valid through the analysis of the acquired ballistocardiogram signal using the second algorithm. For example, the bio-signal measuring device 1000 may extract an entropy value based on the acquired ballistocardiogram signal using the second algorithm, and then determine whether the ballistocardiogram signal is valid based on whether the extracted entropy value satisfies the predetermined criterion.

Illustratively, the bio-signal measuring device 1000 may extract a first indicator of the movement of the user through an analysis of the ballistocardiogram signal using the second algorithm, extract a second indicator of the number of users located on the bio-signal measuring device 1000, and extract a third indicator of the clothes of the user. In this case, the bio-signal measuring device 1000 may determine whether the ballistocardiogram signal acquired using at least one of the first to third indicators is valid.

Referring to FIGS. 11 and 12, when it is determined that the acquired bio-signal is valid, the bio-signal measuring device 1000 may determine whether the placement of the bio-signal measuring device 1000 is appropriate (S1400).

Even when the acquired bio-signal is determined to be valid, a more accurate indicator can be extracted only when the bio-signal is measured in a state where the bio-signal measuring device 1000 is correctly placed. Accordingly, when the target bio-signal is determined to be valid, the bio-signal measuring device 1000 may additionally determine whether the placement of the bio-signal measuring device 1000 is appropriate based on the target bio-signal (S2160).

However, determining whether or not the placement of the bio-signal measuring device 1000 is appropriate is not an essential step and may be selectively performed. For example, determining whether the placement of the bio-signal measuring device 1000 is appropriate may be performed when the user uses the bio-signal measuring device 1000 for the first time. Alternatively, determining whether the placement of the bio-signal measuring device 1000 is appropriate may be performed at every predetermined period (e.g., 1 week, 1 month, etc.).

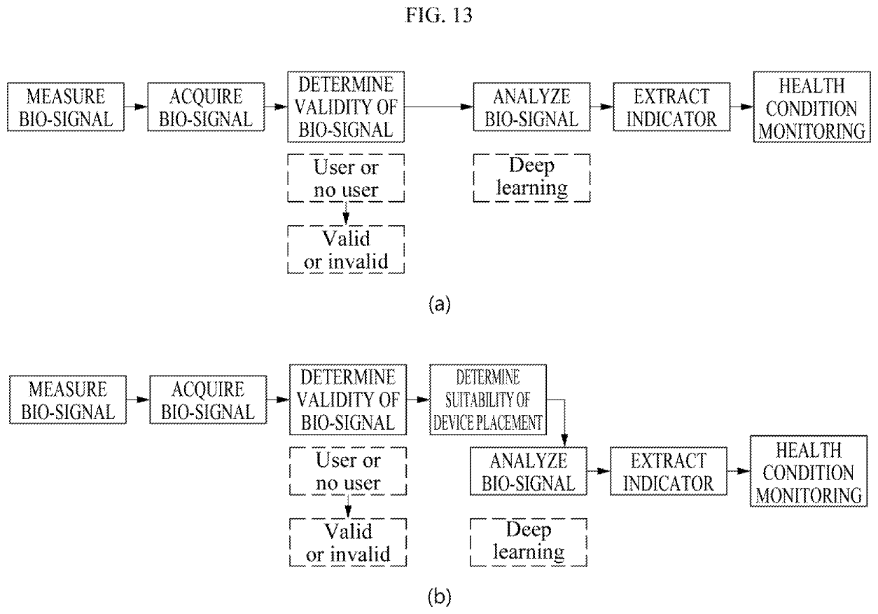

As illustrated in (a) of FIG. 13, the bio-signal measuring device 1000 may perform health condition monitoring while omitting the step of determining whether the placement of the bio-signal measuring device 1000 is appropriate. In addition, as illustrated in (b) of FIG. 13, the bio-signal measuring device 1000 may perform the health condition monitoring while including the determining whether the placement of the bio-signal measuring device 1000 is appropriate.

Hereinafter, a method for determining whether the placement of the bio-signal measuring device 1000 is appropriate will be described in detail with reference to the drawings.

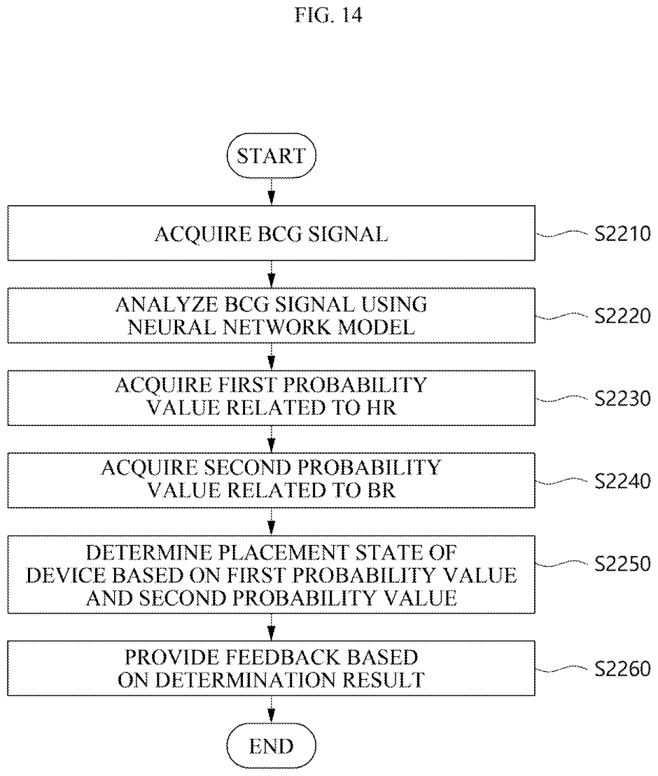

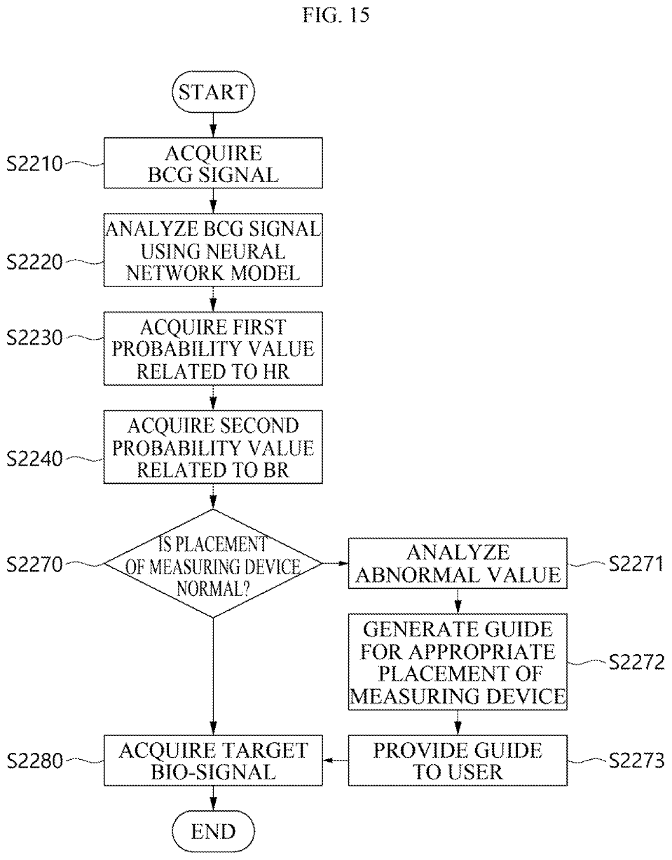

FIGS. 14 and 15 are diagrams for describing a method for determining whether the placement of the bio-signal measuring device is appropriate according to an embodiment.

Referring FIG. 14, the bio-signal measuring device 1000 may perform a step of acquiring the ballistocardiogram signal (S2210), a step of analyzing the ballistocardiogram signal using a neural network model (S2220), a step of acquiring a first probability value related to heart rate (S2230), a step of acquiring a second probability value related to breathing rate (S2240), a step of determining a placement state of the device based on the first probability value and the second probability value (S2250), and a step of providing feedback based on the determination result (S2260).

The bio-signal measuring device 1000 may acquire data about heart rate or breathing rate using a pre-trained neural network model after acquiring the ballistocardiogram signal. The neural network model may be trained to acquire the data about heart rate or breathing rate from the ballistocardiogram signal using a peak detection algorithm.

As an example, the neural network model is a single neural network model and may be trained to acquire data about heart rate and breathing rate by receiving the ballistocardiogram signal. As another example, the neural network model may include a first neural network model trained to acquire the data about heart rate by receiving the ballistocardiogram signal and a second neural network model trained to acquire the data about breathing rate by receiving the ballistocardiogram signal. In this case, the first neural network model and the second neural network model may be physically or logically separated from one another.

The data about heart rate may include a probability value related to heart rate. The probability value related to heart rate may relate to a probability that heart rate is acquired appropriately within a predetermined time period.

The data about breathing rate may include a probability value related to breathing rate. The probability value related to breathing rate may relate to a probability that breathing rate is appropriately acquired within a predetermined time period.

The bio-signal measuring device 1000 may determine whether the bio-signal measuring device 1000 is correctly placed based on the first probability value related to heart rate and the second probability value related to breathing rate acquired from the ballistocardiogram signal.

The bio-signal measuring device 1000 may determine that the bio-signal measuring device 1000 is correctly placed when the first probability value and the second probability value exceed predetermined values. For example, the bio-signal measuring device 1000 may determine that the bio-signal measuring device 1000 is correctly placed when the first probability value and the second probability value exceed a predetermined peak detection probability.

The bio-signal measuring device 1000 determines whether the placement of the bio-signal measuring device 1000 is appropriate based on the first probability value and the second probability value, and then provides feedback to the user based on the determination result.

Referring to FIG. 15, when it is determined that the placement of the bio-signal measuring device 1000 is inappropriate, the bio-signal measuring device 1000 may additionally perform a step of analyzing an abnormal value (S2271), a step of generating a guide for an appropriate placement of the bio-signal measuring device (S2272), and a step of providing the guide to the user (S2273).

When it is determined that the placement of the bio-signal measuring device 1000 is inappropriate, the bio-signal measuring device 1000 may determine the cause of the inappropriate placement of the bio-signal measuring device 1000 based on at least one of a value related to heart rate and a value related to breathing rate extracted from the ballistocardiogram signal. The bio-signal measuring device 1000 may generate a guide for the appropriate placement of the bio-signal measuring device based on at least one of the value related to heart rate and the value related to breathing rate extracted from the ballistocardiogram signal.

For example, the bio-signal measuring device 1000 may generate feedback about a placement position of the bio-signal measuring device 1000 based on at least one of the value related to heart rate and the value related to breathing rate extracted from the ballistocardiogram signal. As a more specific example, the bio-signal measuring device 1000 may generate feedback for guiding the placement of the bio-signal measuring device 1000 to be closer to the upper body part of the user based on at least one of the value related to heart rate and the value related to breathing rate extracted from the ballistocardiogram signal.

As another example, the bio-signal measuring device 1000 may generate feedback according to a placement method for the bio-signal measuring device 1000 based on at least one of the value related to heart rate and the value related to breathing rate extracted from the ballistocardiogram signal. As a more specific example, the bio-signal measuring device 1000, based on at least one of the value related to heart rate and the value related to breathing rate extracted from the ballistocardiogram signal, may generate feedback guiding the leveling of the bio-signal measuring device 1000 or generate feedback guiding removal of a blanket, etc. covering the bio-signal measuring device 1000.

When it is determined that the placement of the bio-signal measuring device 1000 is appropriate, the bio-signal measuring device 1000 may perform a step of acquiring the target bio-signal for health condition monitoring (S2280).

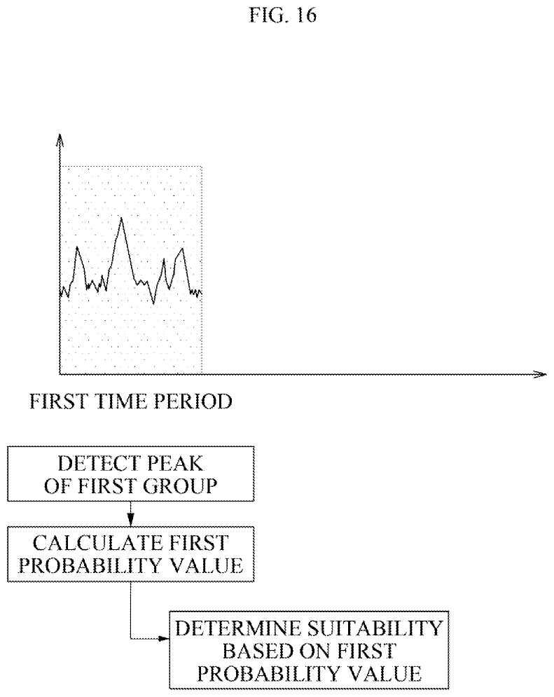

FIGS. 16 to 18 are diagrams for illustratively describing a method for acquiring probability values for heart rate and breathing rate in order to determine whether the placement of the bio-signal measuring device is appropriate according to an embodiment.

The bio-signal measuring device 1000 may repeatedly acquire the ballistocardiogram signal a predetermined number of times during a predetermined time period, and acquire a first probability value related to heart rate and a second probability value related to breathing rate from the ballistocardiogram signal. Thereafter, the bio-signal measuring device 1000 may determine whether the placement of the bio-signal measuring device 1000 is appropriate based on the first probability value and the second probability value acquired by the method described above.

Referring to FIG. 16, the bio-signal measuring device 1000 may acquire a first group of ballistocardiogram signals. The first group may be a set of ballistocardiogram signals acquired during a first predetermined time period.

The bio-signals measuring device 1000 may acquire the first probability value based on the first group of ballistocardiogram signals using a pre-trained neural network model. The first probability value may be a probability value related to a first indicator (e.g., heart rate) and a probability value related to a second indicator (e.g., breathing rate) acquired based on the first group of ballistocardiogram signals.

The bio-signal measuring device 1000 may determine whether the placement of the bio-signal measuring device 1000 is correct based on the first probability value. When it is determined that the first probability value satisfies the predetermined criterion, the bio-signal measuring device 1000 may determine that the placement of the bio-signal measuring device 1000 is appropriate.

The first time period may be set to a predetermined minimum time or more. For example, when the first indicator and the second indicator are extracted from the ballistocardiogram signal, at least one of the first indicator and the second indicator may be extracted based on the ballistocardiogram signal acquired for at least n seconds or more. In this case, the first time period may be determined as a time period of n seconds or more. As a more specific example, when heart rate and breathing rate are extracted from the ballistocardiogram signal, breathing rate may be extracted based on the ballistocardiogram signal acquired for at least 15 seconds. In this case, the first time period may be set to 15 seconds or more.

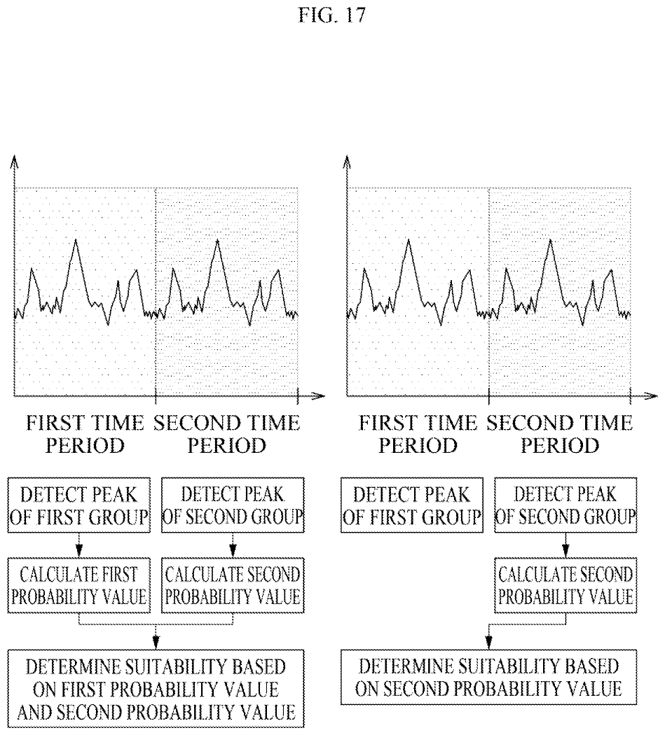

Referring to FIG. 17, the bio-signal measuring device 1000 may acquire a first group of ballistocardiogram signals and a second group of ballistocardiogram signals. The first group may be a set of ballistocardiogram signals acquired during a first predetermined time period, and the second group may be a set of ballistocardiogram signals acquired during a second predetermined time period. In this case, the first time period may be a time period prior to the second time period. For example, the end time point of the first time period may be the same as the start time point of the second time period or may be an earlier time point than the start time point of the second time period.

The bio-signal measuring device 1000, using a pre-trained neural network model, may acquire a first probability value based on the first group of ballistocardiogram signals, and acquire a second probability value based on the second group of ballistocardiogram signals.

The first probability value may be a probability value related to the first indicator (e.g., heart rate) and a probability value related to the second indicator (e.g., breathing rate) acquired based on the first group of ballistocardiogram signals. The second probability value may be a probability value related to the first indicator (e.g., heart rate) and a probability value related to the second indicator (e.g., breathing rate) acquired based on the second group of ballistocardiogram signals.

The bio-signal measuring device 1000 may determine whether the placement of the bio-signal measuring device 1000 is appropriate based on at least one of the first probability value and the second probability value. When it is determined that at least one of the first probability value and the second probability value satisfies the predetermined criterion, the bio-signal measuring device 1000 may determine that the placement of the bio-signal measuring device 1000 is appropriate.

As illustrated in (a) of FIG. 17, the bio-signal measuring device 1000 may determine whether the placement of the bio-signal measuring device 1000 is appropriate based on the first probability value and the second probability value. When it is determined that the first probability value and the second probability value satisfy the predetermined criterion, the bio-signal measuring device 1000 may determine that the placement of the bio-signal measuring device 1000 is appropriate.

As illustrated in (b) of FIG. 17, the bio-signal measuring device 1000 may determine whether the placement of the bio-signal measuring device 1000 is appropriate based on the second probability value. When it is determined that the second probability value satisfies the predetermined criterion, the bio-signal measuring device 1000 may determine that the placement of the bio-signal measuring device 1000 is appropriate.

When the ballistocardiogram signal is acquired through the bio-signal measuring device 1000, since there is a high probability that noise is mixed in the initially acquired ballistocardiogram signal, the bio-signal measuring device 1000 may acquire a probability value related to at least one of heart rate and breathing rate based on the second group of ballistocardiogram signals from which the ballistocardiogram signals are stably acquired. For example, the first group of ballistocardiogram signals are acquired in the first time period. During the first time period, an operation of determining whether the placement of the bio-signal measuring device 1000 is appropriate may be performed by the user. Movements may occur in the bio-signal measuring device 1000 due to the operation of the user. Because of this, the ballistocardiogram signal acquired through the bio-signal measuring device 1000 is mixed with noise, and thus may not be a valid target for analysis through a neural network model. Accordingly, when both the first group of ballistocardiogram signals and the second group of ballistocardiogram signals are acquired, the probability value related to at least one of heart rate and breathing rate may be acquired based on the second group of ballistocardiogram signals.

When the bio-signal measuring device 1000 determines whether the placement of the bio-signal measuring device 1000 is appropriate based on the second group of ballistocardiogram signals, the bio-signal measuring device 1000 may generate feedback about the movement of the user based on the first group of ballistocardiogram signals.

For example, the bio-signal measuring device 1000 may acquire the first probability value related to heart rate and the second probability value related to breathing rate based on the first group of ballistocardiogram signals, and generate feedback about the movement of the user based on at least one of the first probability value and the second probability value. As a more specific example, the bio-signal measuring device 1000 may determine whether the user is currently moving or stationary based on at least one of the first probability value and the second probability value, and, based on this, may generate feedback about the movement of the user.

The bio-signal measuring device 1000 may perform a step of determining the appropriateness of the placement of the bio-signal measuring device 1000 in a state in which the movement of the user is minimized by providing feedback about the movement of the user generated by the method described above to the user, and thus a more accurate determination may be possible.

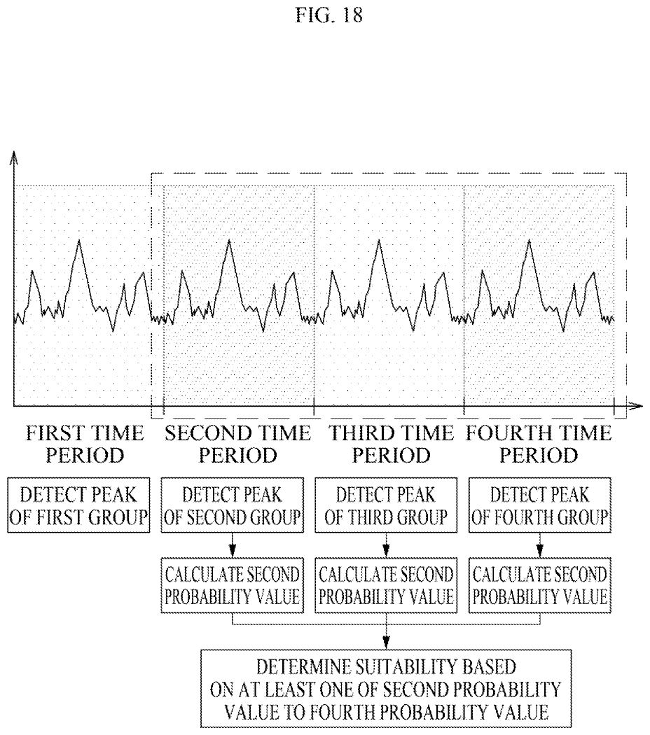

Referring to FIG. 18, the bio-signal measuring device 1000 may acquire the ballistocardiogram signals during a plurality of time periods, and determine whether the placement of the bio-signal measuring device 1000 is appropriate based on the acquired ballistocardiogram signals.

The bio-signals measuring device 1000 may acquire a first group of ballistocardiogram signals, a second group of ballistocardiogram signals, a third group of ballistocardiogram signals, and a fourth group of ballistocardiogram signals. The first group may be a set of ballistocardiogram signals acquired during a first predetermined time period, the second group may be a set of ballistocardiogram signals acquired during a second predetermined time period, the third group may be a set of ballistocardiogram signals acquired during a predetermined third time period, and the fourth group may be a set of ballistocardiogram signals acquired during a fourth predetermined time period.

The bio-signal measuring device 1000, using a pre-trained neural network model, may acquire a first probability value based on the first group of ballistocardiogram signals, acquire a second probability value based on the second group of ballistocardiogram signals, acquire a third probability value based on the ballistocardiogram signals of the third group, and acquire a fourth probability value based on the ballistocardiogram signals of the fourth group.

The first probability value to the fourth probability value are concepts corresponding to the first probability value or the second probability value described with reference to FIGS. 16 and 17, and since the description thereof has been described above, duplicate description thereof will be omitted.

The bio-signal measuring device 1000 may determine whether the placement of the bio-signal measuring device 1000 is appropriate based on at least one of the first probability value, the second probability value, the third probability value, and the fourth probability value. When it is determined that at least one of the first probability value, the second probability value, the third probability value, and the fourth probability value satisfies a predetermined criterion, the bio-signal measuring device 1000 may determine that the placement of the bio-signal measuring device 1000 is appropriate.

The bio-signal measuring device 1000 may determine whether the placement of the bio-signal measuring device 1000 is appropriate based on signals excluding the ballistocardiogram signals acquired in the first time period among the ballistocardiogram signals acquired in the first time period to the fourth time period. For example, the bio-signal measuring device 1000 may determine whether the placement of the bio-signal measuring device 1000 is appropriate based on at least one of the second probability value, the third probability value, and the fourth probability value.

For example, when it is determined that at least one of the second probability value, the third probability value, and the fourth probability value satisfies the predetermined criterion, the bio-signal measuring device 1000 may determine that the placement of the bio-signal measuring device 1000 is appropriate. As another example, when it is determined that all of the second probability value, the third probability value, and the fourth probability value satisfy the predetermined criterion, the bio-signal measuring device 1000 may determine that the placement of the bio-signal measuring device 1000 is appropriate.

The bio-signal measuring device 1000 may determine whether the placement of the bio-signal measuring device 1000 is appropriate, based on the ballistocardiogram signal acquired in the last time period among the ballistocardiogram signals acquired in a plurality of time periods. For example, the bio-signal measuring device 1000 may determine whether the placement of the bio-signal measuring device 1000 is appropriate based on the fourth probability value.

The lengths of the first time period to fourth time period may be equal to each other within an error range. The first to fourth time periods do not overlap each other and may be a continuous time period. Illustratively, the first to fourth time periods may be set to 15 seconds.

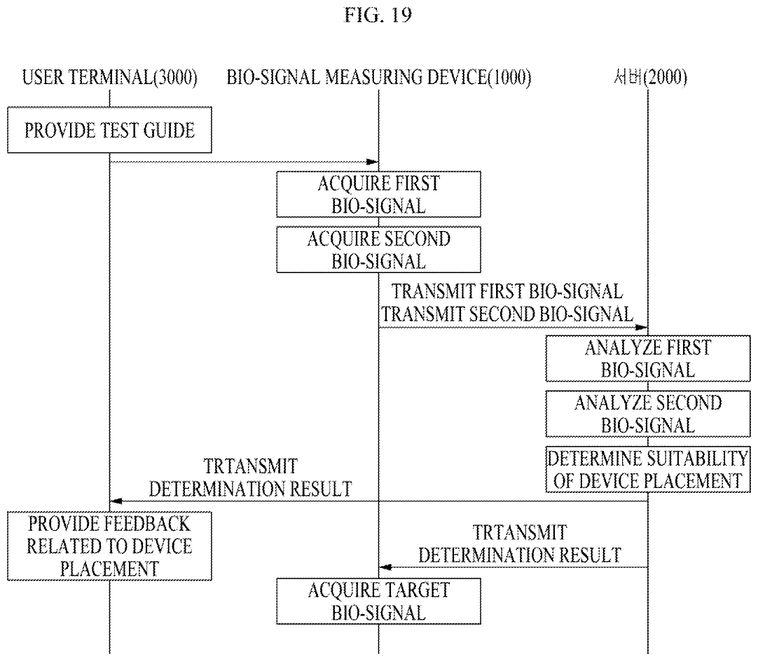

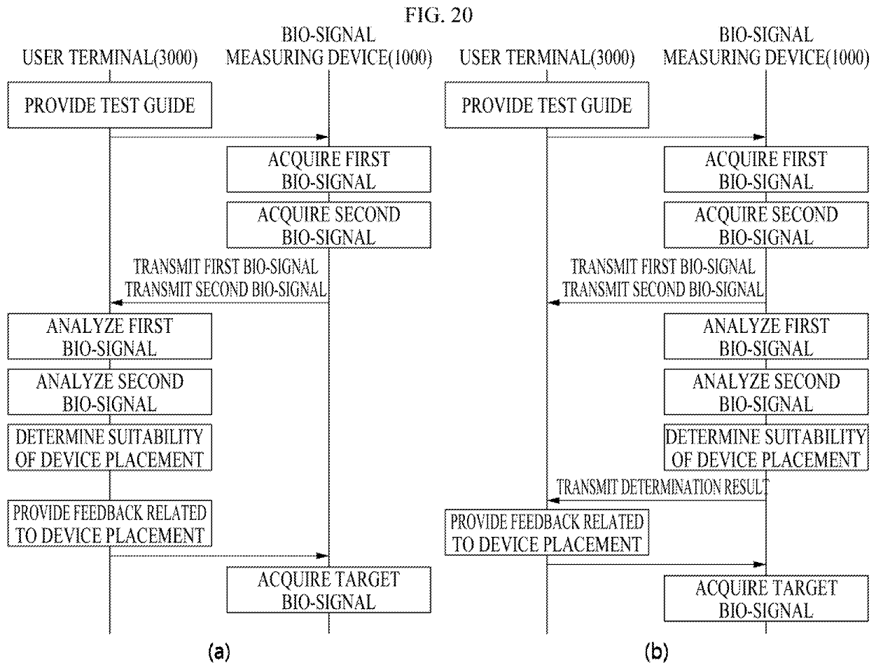

FIGS. 19 and 20 are diagrams for illustratively describing a system for acquiring a target bio-signal according to an embodiment.

Referring to FIG. 19, the system for acquiring the target bio-signal according to an embodiment may include the bio-signal measuring device 1000, the server 2000, and the user terminal 3000.

The user terminal 3000 may provide a test guide to the user. The test guide may be a guide for inducing an operation of the user to determine whether the bio-signal measuring device 1000 is correctly placed or whether the bio-signal measuring device 1000 operates correctly.

The bio-signal measuring device 1000 may acquire at least one bio-signal after acquiring an input of the user for the test guide. For example, the at least one bio-signal may be the ballistocardiogram signal. The bio-signal measuring device 1000 may transmit the at least one acquired bio-signal to the server 2000.

After receiving the at least one bio-signal, the server 2000 may analyze the at least one bio-signal using a pre-trained neural network model. The server 2000 may determine whether the placement of the bio-signal measuring device 1000 is appropriate based on the analysis of the at least one bio-signal. The server 2000 may transmit a result of determining whether the placement of the bio-signal measuring device 1000 is appropriate to the user terminal 3000 or the bio-signal measuring device 1000.

Referring to FIG. 20, the system for acquiring the target bio-signal according to an embodiment may be configured with the bio-signal measuring device 1000 and the user terminal 3000.

At least one bio-signal acquired by the bio-signal measuring device 1000 may be analyzed by the bio-signal measuring device 1000 or the user terminal 3000. For example, as illustrated in (a) of FIG. 20, the user terminal 3000 may analyze at least one bio-signal measured by the bio-signal measuring device 1000 using a pre-trained neural network model, and then determine whether the placement of the bio-signal measuring device 1000 is appropriate based on the analysis result. As another example, as illustrated in (b) of FIG. 20, the bio-signal measuring device 1000 may analyze at least one acquired bio-signal using a pre-trained neural network model, and then determine whether the placement of the bio-signal measuring device 1000 is appropriate based on the analysis result.

4. Bio-Signal Measuring Device

As described above, the bio-signal measuring device 1000 may acquire at least one bio-signal generated from the human body during sleep.

Conventionally, in order to acquire the bio-signals generated from the human body during sleep, the user had to sleep while wearing a wearable device. Conventional devices have low user convenience in that the bio-signals can be acquired only when the user wears the wearable device even during sleep.

The bio-signal measuring device 1000 according to an embodiment of the present application can acquire the bio-signals even when the user is not directly wearing the device, and can acquire the bio-signals with high accuracy even when the bio-signals are acquired through indirect contact.

The bio-signal measuring device 1000 according to an embodiment may be provided in various forms depending on a hardware structure, a type of bio-signal to be measured, and a method for measuring the bio-signal.

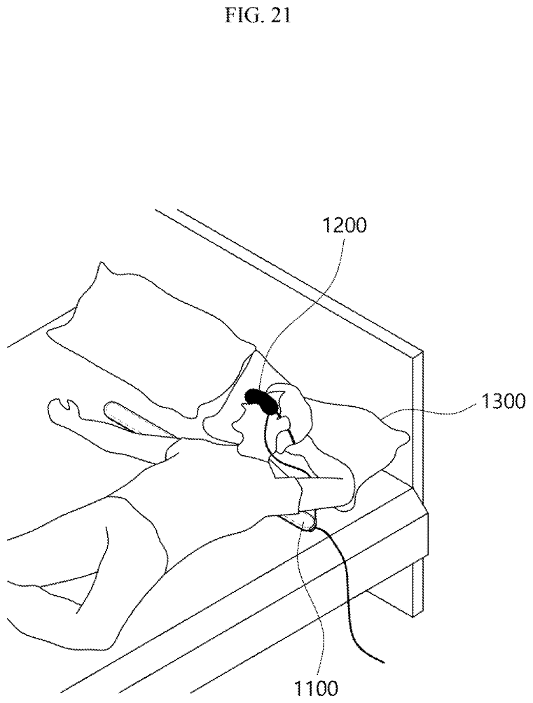

FIG. 21 is a diagram for illustratively describing the bio-signal measuring device according to an exemplary embodiment. Referring to FIG. 21, the bio-signal measuring device 1000 may include a first bio-signal measuring device 1100, a second bio-signal measuring device 1200 and a third bio-signal measuring device 1300.

The first bio-signal measuring device 1100 to the third bio-signal measuring device 1300 may operate in conjunction with each other.

For example, the server 2000 may monitor the health condition using a first bio-signal acquired through the first bio-signal measuring device 1100 and a second bio-signal acquired through the second bio-signal measuring device 1200. In this case, the first bio-signal and the second bio-signal may be identical to each other.

As another example, the server 2000 may perform health condition monitoring based on the bio-signal acquired through the first bio-signal measuring device 1100, and provide a notification to the user through the second bio-signal measuring device 1200 based on a result of the health condition monitoring.

The server 2000 does not have to use all of the first bio-signal measuring device 1100 to the third bio-signal measuring device 1300 to perform the health condition monitoring. The server 2000 may perform the health condition monitoring using at least one of the first bio-signal measuring device 1100 to the third bio-signal measuring device 1300.

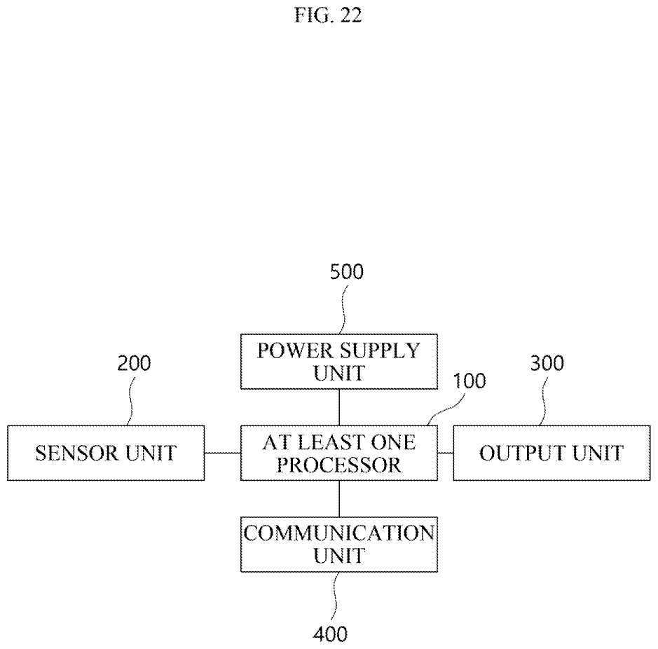

FIG. 22 is a diagram for describing a configuration of the bio-signal measuring device according to an embodiment. Referring to FIG. 22, the bio-signal measuring device 1000 according to an embodiment may include at least one processor 100, a sensor unit 200, an output unit 300, a communication unit 400, and a power supply unit 500.

The sensor unit 200 may include at least one sensor. The sensor unit 200 may measure various bio-signals generated from the human body using the at least one sensor according to a control command of the at least one processor 100. For example, the sensor unit 200 may include at least one of a force sensor, a sound sensor, a temperature sensor, a humidity sensor, a gyro sensor, a motion sensor, a touch sensor, and a proximity sensor. However, the types of sensors that may be included in the sensor unit 200 are not limited thereto, and various known types of sensors for measuring the bio-signals generated from the human body may be included.

The output unit 300 may output various types of alarms according to the control command of the at least one processor 100. For example, the output unit 300 may provide an alarm to the user using a vibration module according to the control command of the at least one processor 100. As another example, the output unit 300 may provide the alarm to the user using a speaker according to the control command of the at least one processor 100. As still another example, the output unit 300 may provide the alarm to the user with using an LED according to the control command of at least one processor 100. As still another example, the output unit 300 may output information related to a health condition or sleep stage of the user through a display panel.

The communication unit 400 may include a wireless communication module and/or a wired communication module. Here, the wireless communication module may include a Wi-Fi communication module, a cellular communication module, and the like.

The power supply unit 500 includes a battery, and the battery may be built into the user terminal 1000 or provided to be detachable from the outside. The power supply unit 600 may supply power required by each component of the user terminal 1000.

The at least one processor 100 may execute a predetermined operation by executing at least one command stored in a memory. More specifically, the at least one processor 100 may control overall operations of components included in the bio-signal measuring device 1000.

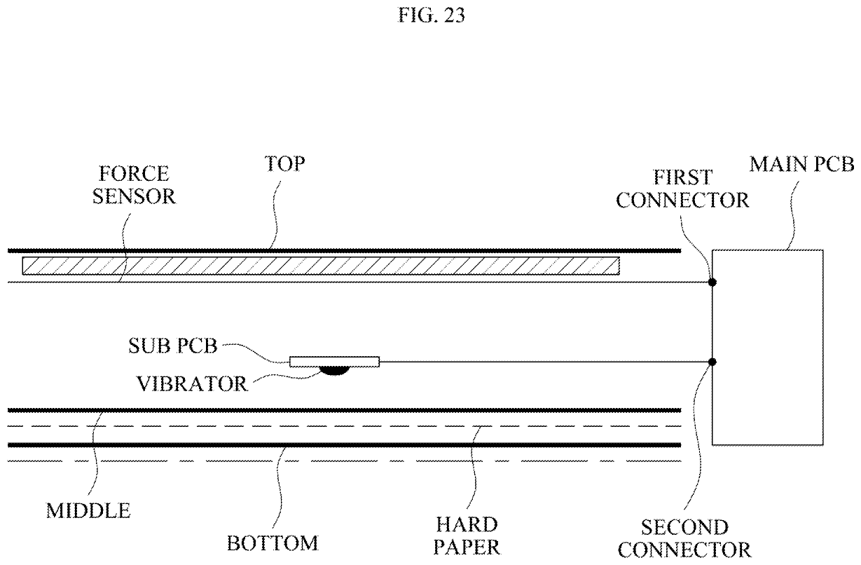

FIG. 23 is a diagram for illustratively describing a structure of the measuring device according to an embodiment.

Referring to FIG. 23, the first bio-signal measuring device 1100 may have a structure for measuring the bio-signal generated from the upper body part of the human body. For example, the first bio-signal measuring device 1100 may be provided to be placed on a mattress of a bed to measure the ballistocardiogram signal generated from the upper body part of the user.

The first bio-signal measuring device 1100 may include at least one force sensor. The at least one force sensor may be formed in a longitudinal direction.

The force sensor may have one surface and another surface. The one surface may be in a direction toward the human body when the first bio-signal measuring device 1100 is placed on the mattress, and the other surface may be in a direction toward the mattress when the first bio-signal measuring device 1100 is placed on the mattress.

The force sensor may be attached to a first cover (top) to be described later. One surface of the force sensor may be attached to the first cover (top).

The first bio-signal measuring device 1100 may include a plurality of covers. The first bio-signal measuring device 1100 may include the first cover (top), a second cover (middle), and a third cover (bottom). Meanwhile, the first bio-signal measuring device 1100 essentially includes the first cover (top) and the third cover (bottom), but may selectably include the second cover (middle).

When the second cover (middle) is additionally included in the first bio-signal measuring device 1100, the weight of the first bio-signal measuring device 1100 is increased, and accordingly, the first bio-signal measuring device 1100 can be less affected by the movement of the user.

The first cover (top) to the third cover (bottom) may be provided to be formed of a fabric. Alternatively, the first cover (top) to the third cover (bottom) may be provided to be formed of a plastic material. However, this is exemplary, and the materials of the first cover (top) to the third cover (bottom) are not limited thereto, and may be provided to be formed of various known materials.

The first cover (top) to the third cover (bottom) may be waterproofed. The first cover (top) to the third cover (bottom) may be subjected to coating treatment with waterproofing performance. Since the first bio-signal measuring device 1100 is a device for acquiring the bio-signal generated from the user during sleep, the first bio-signal measuring device 1100 is highly likely to be exposed to moisture such as sweat generated during sleep. In this case, the waterproof function is applied to the first cover (top) to the third cover (bottom) so that various members placed between the first cover (top) and the third cover (bottom) can operate safely without being exposed to moisture.

An anti-slip coating may be additionally formed on the lower surface of the third cover (bottom). The lower surface of the third cover (bottom) may come into contact with the mattress of the bed. The anti-slip coating is additionally formed on the lower surface of the third cover (bottom) so that the first bio-signal measurement 1100 can be less affected by the movement of the user.

The first cover (top) may be formed to cover at least a portion of a region corresponding to the one surface of the force sensor, and the third cover (bottom) may be formed to cover at least a portion of a region corresponding to the other surface of the force sensor.

The third cover (bottom) may be formed to correspond to a shape of the first cover (top). When the third cover (bottom) and the first cover (top) are coupled, an internal space may be formed, and members such as the force sensor and a vibrator may be placed in the internal space.

The first bio-signal measuring device 1100 may include hard paper placed between the third cover (bottom) and the other surface of the force sensor. The first bio-signal measuring device 1100 may include hard paper placed between the first cover (top) and the third cover (bottom).

The hard paper may be placed between the third cover (bottom) and the other surface of the force sensor to minimize the movement of the force sensor and the vibrator caused by the movement of the user. More specifically, when the user sleeps on the first bio-signal measuring device 1100, due to the movement of the user, movement may also occur in the first bio-signal measuring device 1100 and components constituting the first bio-signal measuring device 1100. Due to such movement, the accuracy of the bio-signal measured through the first bio-signal measuring device 1000 may be lowered, and thus, the movement of the first bio-signal measuring device 1000 and components constituting the first bio-signal measuring device 1100 needs to be minimized. In this case, the hard paper may be placed inside the first bio-signal measuring device to perform a function of minimizing movement of the force sensor and the vibrator, etc. generated by the movement of the user. Furthermore, the material of the hard paper comprise a variety of materials known in the art, and may be changed to another material without being limited to any one of the various materials known in the art.

The first bio-signal measuring device 1100 may include at least one vibrator placed between the hard paper and the other surface of the force sensor. In the drawings, it is illustrated that the first bio-signal measuring device 1100 includes one vibrator, but is not limited thereto, and the first bio-signal measuring device 1100 may include a plurality of vibrators.

When the plurality of vibrators are provided in the first bio-signal measuring device 1100, each vibrator may be placed to have a regular interval. When the plurality of vibrators are provided in the first bio-signal measuring device 1100, each vibrator may be placed to have an interval that gradually narrows toward the center. However, when the plurality of vibrators are provided in the first bio-signal measuring device 1100, a method for placing each vibrator may be varied from this configuration.

The vibrator may be placed on a SUB-PCB. The vibrator may be placed on the SUB-PCB in a direction toward the third cover (bottom). The vibrator may be placed on the SUB-PCB in a direction in which the other surface of the force sensor faces. When the vibrator is placed in a direction toward the third cover (bottom), more effective vibration may be provided to the human body. When the vibrator is placed in the direction toward the third cover (bottom), it is possible to minimize the presence of a hard vibrator as felt by the user when the user is lying on top of the device (as the edges of the hard vibrator are cushioned by the mattress below). In addition, when the vibrator is placed in the direction toward the third cover (bottom), an accuracy of bio-signal measurement through the force sensor may be further improved. More specifically, when the vibrator is placed in a direction toward the first cover (top), since the force sensor is directly affected by vibration of the vibrator, the accuracy of the measured bio-signal may be lowered compared to the case where the vibrator is placed in the direction toward the third cover (bottom).

The vibrator may be placed on the SUB-PCB in the direction toward the first cover (top). In this case, vibration generated from the vibrator may be more directly transmitted to the human body.

The first bio-signal measuring device 1100 may include a MAIN-PCB. The MAIN-PCB may be electrically connected to the force sensor and the vibrator. The MAIN-PCB may be electrically connected to the force sensor through a first connector and electrically connected to the vibrator through a second connector.

Since the force sensor is formed long in the length direction and is placed between the first cover (top) and the third cover (bottom), when the force sensor is electrically connected to the MAIN-PCB through a connector separate from the vibrator, the force sensor can be more stably placed.

In addition, when the force sensor and the vibrator are electrically connected to the MAIN-PCB through separate connectors, respectively, it may be easier to manufacture the first bio-signal measuring device 1100.

According to one embodiment, the first bio-signal measuring device 1100 may further include a housing capable of accommodating the MAIN-PCB. A force sensor accommodating region contacting at least a portion of the force sensor may be provided in the housing. The first bio-signal measuring device 1100 may further include a cover covering the force sensor accommodating region.

The force sensor accommodating region may be formed to be inclined at a predetermined angle. As the force sensor accommodating region is formed to be inclined at a predetermined angle, a region in which the force sensor contacts the force sensor accommodating region is increased so that the force sensor may be more stably inserted and placed in the housing.

According to another embodiment, the first cover (top) and the third cover (bottom) of the first bio-signal measuring device 1100 may be formed to extend to surround at least a portion of the MAIN-PCB. The first bio-signal measuring device 1100 may accommodate the MAIN-PCB using the first cover (top) and the third cover (bottom) without including a separate housing for accommodating the MAIN-PCB.

The first cover (top) and the third cover (bottom) are formed to surround different surfaces of the MAIN-PCB, so that the MAIN-PCB may be placed between the first cover (top) and the third cover (bottom). That is, the first cover (top) may be formed to extend to surround at least a portion of a region corresponding to one surface of the MAIN-PCB, and the third cover (bottom) may be formed to extend to surround at least a portion of a region corresponding to the other surface of the MAIN-PCB.

On the other hand, even when the first bio-signal measuring device 1100 further includes the housing capable of accommodating the MAIN-PCB according to an embodiment, the housing may be integrally formed with the first cover (top) and the third cover (bottom).

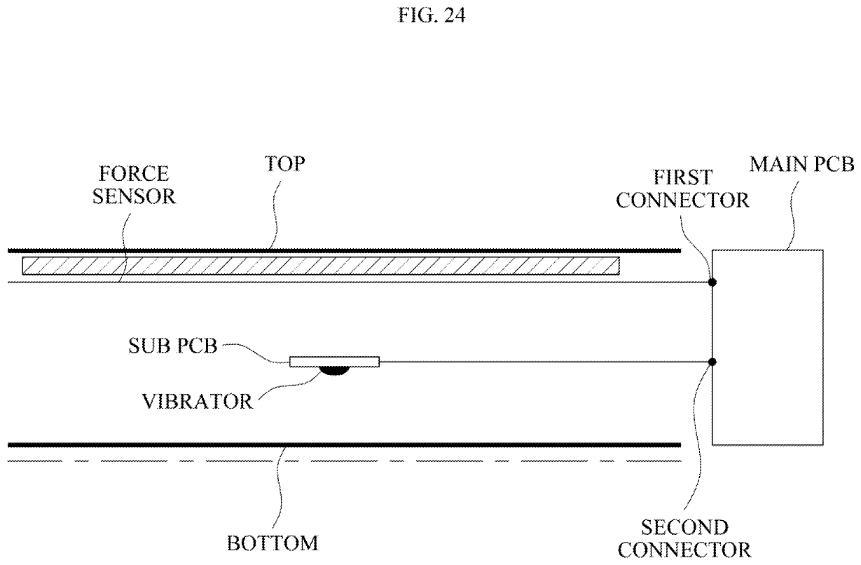

FIG. 24 is a diagram for illustratively describing the structure of a first bio-signal measuring device according to another embodiment.

Referring to FIG. 24, the first bio-signal measuring device 1100 according to another embodiment may include the force sensor, and the first cover (top) may be placed on one surface of the force sensor, and the third cover (bottom) may be placed on the other surface of the force sensor. The vibrator placed on the SUB-PCB may be provided between the other surface of the force sensor and the third cover (bottom). The first bio-signal measuring device 1100 may include a MAIN-PCB electrically connected to the force sensor through the first connector and electrically connected to the vibrator through the second connector, and a housing accommodating the MAIN-PCB.

Compared to the first bio-signal measuring device 1100 according to an embodiment described with reference to FIG. 23, the first bio-signal measuring device 1100 according to another embodiment may be provided with the second cover (middle) and hard paper (hard paper) omitted. In this case, a material of the first cover (top) and the third cover (bottom) may be a hard material such as plastic.

When the material of the first cover (top) and the third cover (bottom) is provided to be formed of the hard material such as plastic, the first bio-signal measuring device 1100 can operate with minimal influence from the movement of the user even without being provided with an additional configuration such as the second cover (middle) or hard paper.

Meanwhile, components constituting the first bio-signal measuring device 1100 according to another embodiment have the same or corresponding structure as the components described with reference to FIG. 23, and thus duplicate description thereof will be omitted.

The second bio-signal measuring device 1200 may have a structure for measuring the bio-signal generated in a part around the of the human body. The second bio-signal measuring device 1200 may directly contact at least a portion of the human body. For example, the second bio-signal measuring device 1200 is provided in the form of an eye patch and can measure the bio-signals generated in the part around the eye of the human body. The bio-signal generated in the region around the eye may be at least one of the ballistocardiogram signal, the sound signal, temperature, and humidity.

The third bio-signal measuring device 1300 may have a structure for measuring the bio-signal generated from the head of the human body. The third bio-signal measuring device 1300 may directly contact at least a portion of the human body. For example, the third bio-signal measuring device 1300 is provided in the form of a pillow and can measure the bio-signal generated from the head of the human body. The bio-signal generated from the head may be at least one of the ballistocardiogram signal, the sound signal, temperature, and humidity.

As a more specific example, the force sensor may be provided in the second bio-signal measuring device 1200. The second bio-signal measuring device 1200 may measure a temporal pulse generated in the temple using the force sensor.

As another example, an electrooculography (EOG) sensor may be provided in the second bio-signal measuring device 1200. The second bio-signal measuring device 1200 may measure the electrooculography using the EOG measurement sensor.

As another example, a sound sensor may be provided in the second bio-signal measuring device 1200, and the second bio-signal measuring device 1200 may acquire a sound signal generated from the human body during sleep using the sound sensor.

As another example, a temperature sensor or a humidity sensor may be provided in the second bio-signal measuring device 1200, and the second bio-signal measuring device 1200 may acquire data about skin temperature or humidity using the temperature sensor or the humidity sensor.

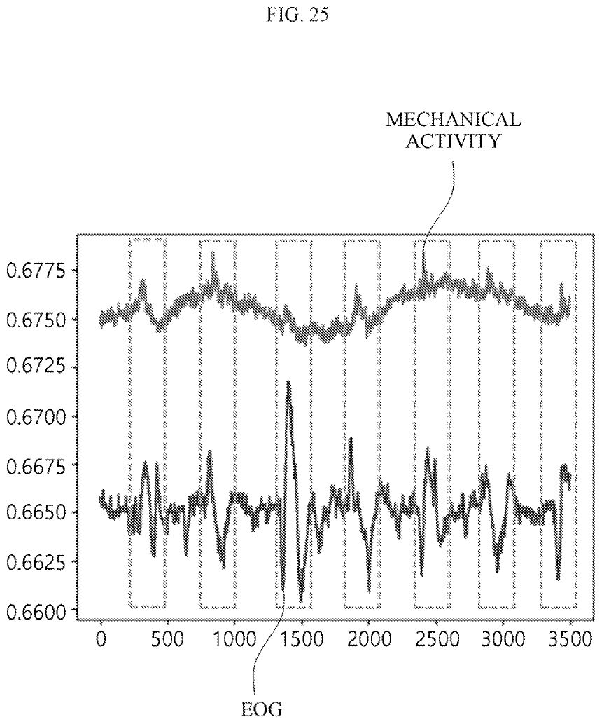

FIGS. 25 to 27 are diagrams for illustratively describing a method for monitoring the sleep stage based on a pressure value around the eye by the bio-signal measuring device according to an embodiment.

In general, the electrooculograph measured through the electrooculography (EOG) measurement sensor is used to determine the movement of the eye. However, since a separate and complicated device is additionally required to measure the electrooculography, this method is not suitable for determining the movement of the eye observed during sleep. In addition, in the existing electrooculography measuring device, user convenience was low because sticky electrodes were used, and even when dry electrodes were used, contact was not well maintained, and thus there is a limitation in that it is difficult to continuously measure the bio-signals during sleep. Furthermore, there is also a limitation in that measurements should be made at at least two or more points on the body in order to measure the existing electrooculography.

The bio-signal measuring device 1000 according to an embodiment of the present application may determine the movement of the eye using the pressure value around the eye, and monitor the sleep stage based on this.

Referring to FIG. 25, a mechanical activity signal acquired based on a pressure value around the eye and a electrooculography (EOG) signal acquired through the electrooculography (EOG) measurement sensor may be checked. Each of the mechanical activity signal and the electrooculography (EOG) signal is acquired for the same period of time by using the same person as the subject.

It can be checked that the waveform of the mechanical activity and the waveform of the electrooculography (EOG) signal correspond to each other. In other words, it is possible to acquire information about the movement of the eye, based on the pressure value measured around the eye. As a result, it is possible to determine the movement of the eye based on the pressure value measured around the eye.

Hereinafter, with reference to the drawings, a method in which the bio-signal measuring apparatus 1000 according to an embodiment monitors the movement of the eye and the sleep stage using the pressure value around the eye.

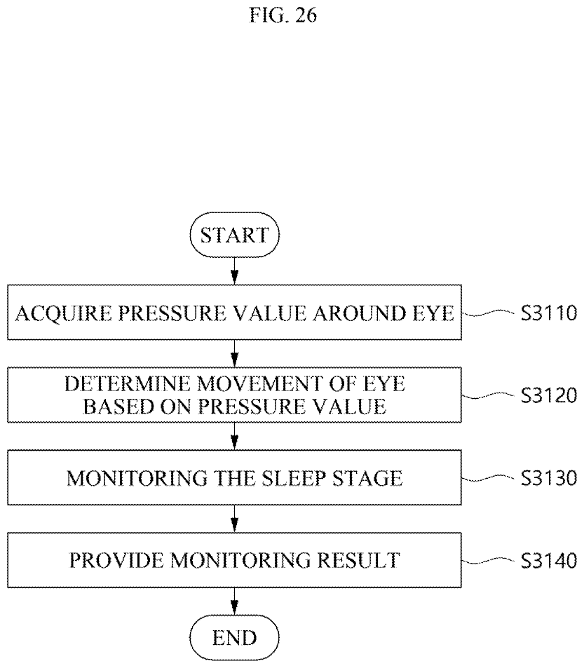

Referring to FIG. 26, the bio-signal measuring device 1000 may perform a step of acquiring the pressure value around the eye (S3110), a step of determining the movement of the eye based on the pressure value (S3120), a step of monitoring the sleep stage (S3130), and a step of providing the monitoring result (S3140).

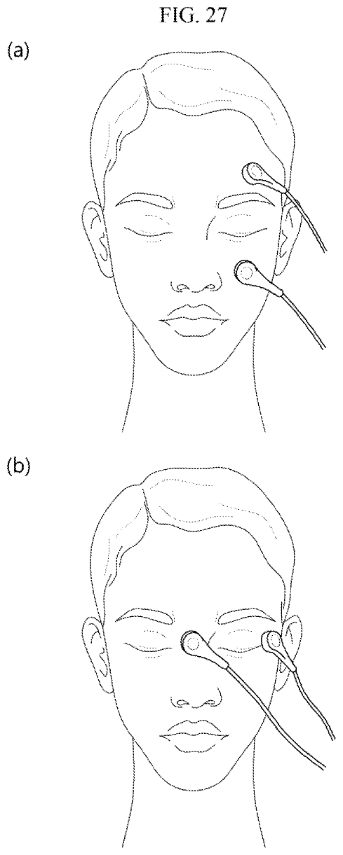

Referring to FIG. 27, the bio-signal measuring device 1000 may acquire the pressure value around the eye using at least one force sensor. The bio-signal measuring device 1000 may acquire the temporal pulse generated in the temple using at least one force sensor.

As illustrated in (a) of FIG. 27, the bio-signal measuring device 1000 may include the force sensor in a region corresponding to the upper side or lower side of the eye. In (a) of FIG. 27, the bio-signal measuring device 1000 is illustrated as having force sensors on the upper side and lower side of the eye, respectively, but is not limited thereto. The bio-signal measuring device 1000 may include the force sensor only on one of the upper side and lower side of the eye. In this case, the bio-signal measuring device 1000 may acquire a pressure value generated above and below the eye.

As illustrated in (b) of FIG. 27, the bio-signal measuring device 1000 may include a force sensor in a region corresponding to the left side or right side of the eye. In (b) of FIG. 27, the bio-signal measuring device 1000 is illustrated as having force sensors on the left side and right side of the eye, but is not limited thereto. The bio-signal measuring device 1000 may include the force sensor only on one of the left side and right side of the eye. In this case, the bio-signal measuring device 1000 may acquire the pressure value generated from the left side and right side of the eye.

In addition, although not illustrated in the drawings, the bio-signal measuring device 1000 may include the force sensor on at least one of the upper side and lower side of the eye and the force sensor on at least one of the left side and right side of the eye. In this case, the bio-signal measuring device 1000 may acquire all pressure values generated from the upper side and lower side of the eye or left side and right side of the eye.

When the bio-signal measuring device 1000 monitors the sleep stage using the pressure value around the eye, the bio-signal measuring device 1000 may be provided in the form of a wearable device. When the bio-signal measuring device 1000 is the wearable device, the force sensor may directly or indirectly contact the body, and may be provided in a fixed form in close contact with the body.

The biological signal measuring device 1000 may determine the movement of the eye based on the pressure value generated around the eye acquired by the method described above, and may monitor the sleep stage based on the determination result. Hereinafter, a method for monitoring, by the bio-signal measuring device 1000, the movement of the eye and the sleep stage based on the pressure value around the eye, will be described with reference to the accompanying drawings.

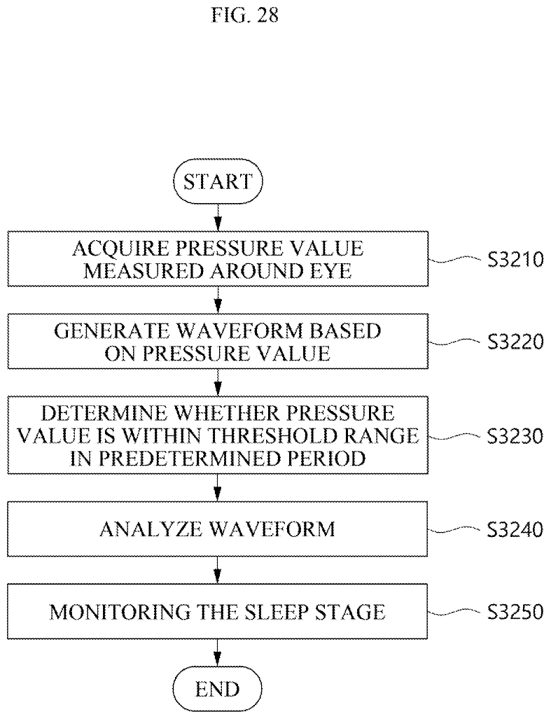

FIGS. 28 to 30 are diagrams for illustratively describing a method for determining the movement of the eye and a method for monitoring the sleep stage by the bio-signal measuring device according to an embodiment.

Referring to FIG. 28, the bio-signal measuring device 1000 according to an embodiment may include a step of acquiring the pressure value measured around the eye (S3210), a step of generating a waveform based on the pressure value (S3220), a step of determining whether the pressure value is within a threshold range in a predetermined period (S3230), a step of analyzing the waveform (S3240), and a step of monitoring the sleep stage (S3250).

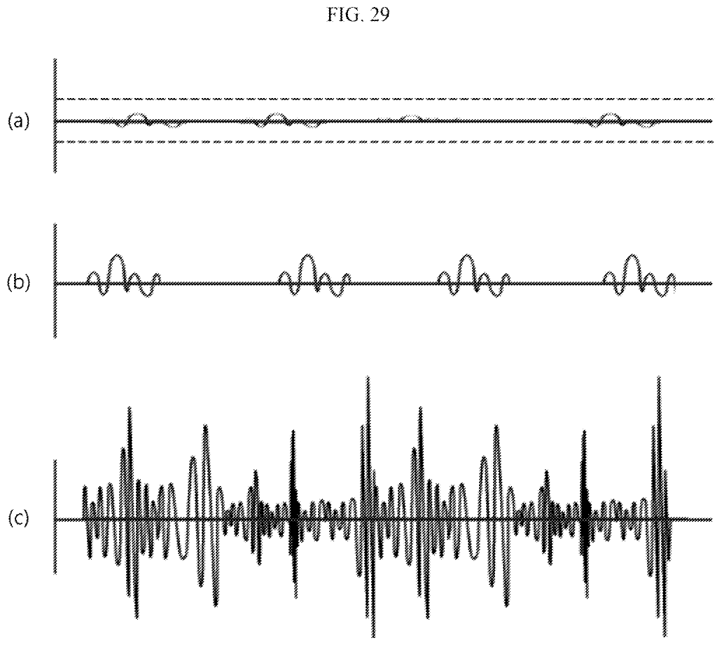

The bio-signal measuring device 1000 may acquire the pressure value generated around the eye and generate a waveform based on the pressure value. The type of waveform generated based on the pressure value measured around the eye may vary. The type of waveform may include a first type of waveform as illustrated in (a) of FIG. 29, a second type of waveform as illustrated in (b) of FIG. 29, and a third type of waveform as illustrated in (c) of FIG. 29.

The bio-signal measuring device 1000 may determine whether the acquired pressure value is within the threshold range in the predetermined period, and monitor the sleep stage through an analysis of the waveform.

The analysis of the waveform may include an analysis of a pattern of the waveform, an analysis of change in the waveform, an analysis of a peak caused by the waveform, analysis of the magnitude of amplitude caused by the waveform, which may be performed through a predetermined algorithm.

According to an embodiment, the bio-signal measuring device 1000 may determine a phase of sleep based on an acquired pressure value and the analysis of the waveform generated based on pressure. The bio-signal measuring device 1000 may determine the phase of sleep according to the acquired pressure value and the type of waveform (e.g., a first type, second type, and third type) generated based on the pressure.

The first type to third type may be determined according to whether a shape of the waveform generated based on the pressure has regularity. For example, the first type may have a relatively regular shape of the generated waveform compared to the second type, and the second type may have a relatively regular shape of the generated waveform compared to the third type.

In this case, the bio-signal measuring device 1000 may determine that the sleep stage of the user is in a first state when the acquired pressure value is within the threshold range in the predetermined period and the waveform is of the first type. In this case, the first state may be a light sleep or deep sleep stage.

As another example, the bio-signal measuring device 1000 may determine that the sleep stage of the user is in a second state when the acquired pressure value exceeds the threshold range in the predetermined period, and the waveform is of the second type. The bio-signal measuring device 1000 may determine that the sleep stage of the user is in the second state when the acquired pressure value exceeds the threshold range in the predetermined period and the waveform maintains a constant pattern within an allowable error range. In this time, the second state may be a wake state.

As another example, the bio-signal measuring device 1000 may determine that the sleep stage of the user is in the third state when the acquired pressure value exceeds the threshold range in the predetermined period and the waveform is of the third type. The bio-signal measuring device 1000 may determine that the sleep stage of the user is in the third state when the acquired pressure value exceeds the threshold range in the predetermined period, and the waveform is irregular or a peak outside a threshold value is found above a predetermined criterion in the waveform. In this case, the third state may be a rapid movement of eye (REM) sleep stage.

The first type to third type may be determined according to the magnitude of the amplitude of the waveform generated based on pressure. For example, the type of the waveform may be determined as a first type when the magnitude of the amplitude is first magnitude, may be determined as a second type when the magnitude of the amplitude is second magnitude, and may be determined as a third type when the magnitude of the amplitude is third magnitude. The second magnitude may be larger than the first magnitude, and the third magnitude may be larger than the second magnitude.

In this case, the bio-signal measuring device 1000 may determine that the sleep stage of the user is in a non-REM sleep stage (e.g., light sleep stage or deep sleep stage) when the acquired pressure value is within a threshold range in a predetermined period and the magnitude of the amplitude of the waveform is the first magnitude.

As another example, the bio-signal measuring device 1000 may determine that the user is in the wake state when the acquired pressure value exceeds the threshold range in the predetermined period, and the magnitude of the amplitude of the waveform is the second magnitude, and may determine that the user is in the REM sleep stage when the magnitude of the amplitude of the waveform is the third magnitude. In this case, the third magnitude may be larger than the second magnitude.

According to another embodiment, the bio-signal measuring device 1000 may determine the phase of sleep based on the pressure value around the eye, but may determine the phase of sleep by additionally considering a reference value.

The bio-signal measuring device 1000 may determine the reference value based on the pressure value around the eye measured while the user is in a wake state. The bio-signal measuring device 1000 may determine the reference value based on a pressure value measured for a predetermined amount of time (e.g., several seconds) from a first time point when the measurement of the pressure value around the eye was started. The bio-signal measuring device 1000 may determine the reference value based on a pressure value measured for a predetermined amount of time in the past (e.g., several seconds) from a second time point when the measurement of the pressure value around the eye ends.

Since the reference value is the pressure value around the eye measured while the user is in a wake state, the bio-signal measuring device 1000 may determine whether the user is in the wake state through the reference value, and may determine the phase of sleep by considering this.

As a more specific example, the bio-signal measuring device 1000 may compare the acquired pressure value with the reference value to predict the phase of sleep of the user. The bio-signal measuring device 1000 may predict the phase of sleep of the user by considering the difference between the acquired pressure value and the reference value. The bio-signal measuring device 1000 may determine whether the user is in the wake state or the sleeping state (e.g., REM sleep or non-REM sleep) by considering the difference between the acquired pressure value and the reference value.

The bio-signal measuring device 1000 may determine that the user is in the REM sleep stage when the difference between the acquired pressure value and the reference value satisfies the first range, and may determine that the user is in the non-REM sleep stage (e.g., a light sleep stage or a deep sleep stage) when the difference between the acquired pressure value and the reference value satisfies the second range. In this case, the first range may be greater than the second range.

Meanwhile, the bio-signal measuring device 1000 may determine a threshold range of the acquired pressure value based on the reference value described with reference to FIG. 28. The bio-signal measuring device 1000 may determine whether the pressure value acquired based on the reference value is within the threshold range or exceeds the threshold range in the predetermined period. Since the pressure value measured around the eye may vary depending on the measurement method or the user, the sleep stage can be more accurately monitored by deciding the reference value in advance and determining the threshold range based on the reference value.

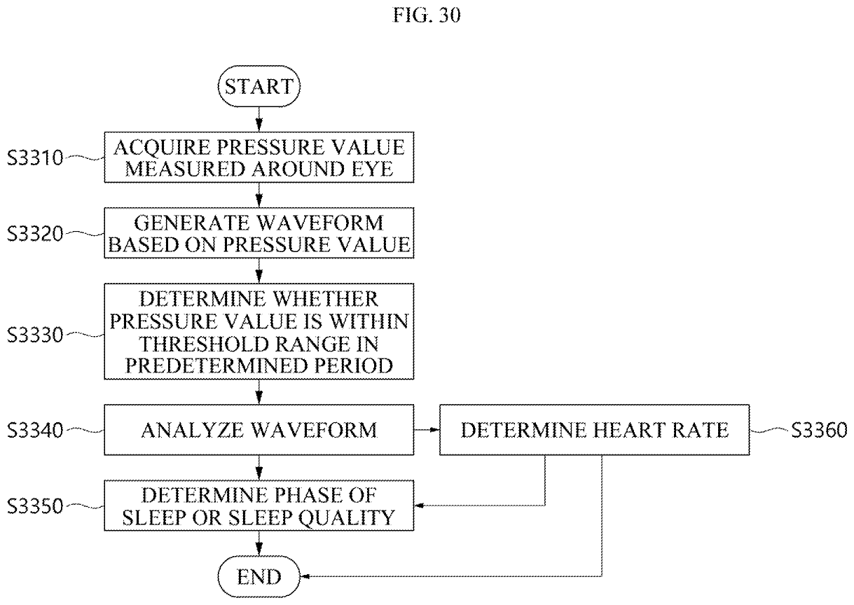

Referring to FIG. 30, the bio-signal measuring device 1000 according to an embodiment may additionally perform a step of determining heart rate through analysis of the waveform (S3360). The bio-signal measuring device 1000 may acquire the pressure value around the eye, generate a waveform based on the acquired pressure value, and determine heart rate of the user using the generated waveform.

The bio-signal measuring device 1000 may acquire heart rate of the user based on the pressure value around the eye through a pre-trained neural network model. The neural network model may be trained to acquire data about heart rate based on training data related to the pressure value around the eye.

The bio-signal measuring device 1000 can monitor the sleep stage more accurately by additionally acquiring information about heart rate of the user as well as information about the movement of eye based on the pressure value around the eye.

For example, the bio-signal measuring device 1000 may acquire first information about the movement of eye based on the pressure value around the eye, and acquire second information about heart rate based on the pressure value around the eye. The bio-signal measuring device 1000 may perform sleep stage monitoring based on the first information and the second information. The bio-signal measuring device 1000 may predict the phase of sleep, and determine sleep quality based on the first information and the second information.

As another example, the bio-signal measuring device 1000 may acquire first information about the movement of eye based on the pressure value around the eye, and determine an expected phase of sleep of the user based on the first information. After acquiring the second information about heart rate based on the pressure value around the eye, the bio-signal measuring device 1000 may monitor the sleep stage of the user or determine sleep quality of the user based on the expected phase of sleep of the user and the second information.

The bio-signal measuring device 1000 may provide feedback to the user based on the result of the sleep stage monitoring of the user determined by the method described above.

The bio-signal measuring device 1000 may include an output unit, and may provide various types of alarms (e.g., sound alarm using a speaker, visual alarm using an LED, etc.) to the user through the output unit based on the result of the sleep stage monitoring of the user.

The bio-signal measuring device 1000 may include a communication unit, and may transmit an electronic signal through the communication unit so that an external device (e.g., a user terminal, other electronic devices, etc.) provides an alarm to the user based on the result of the sleep stage monitoring of the user.

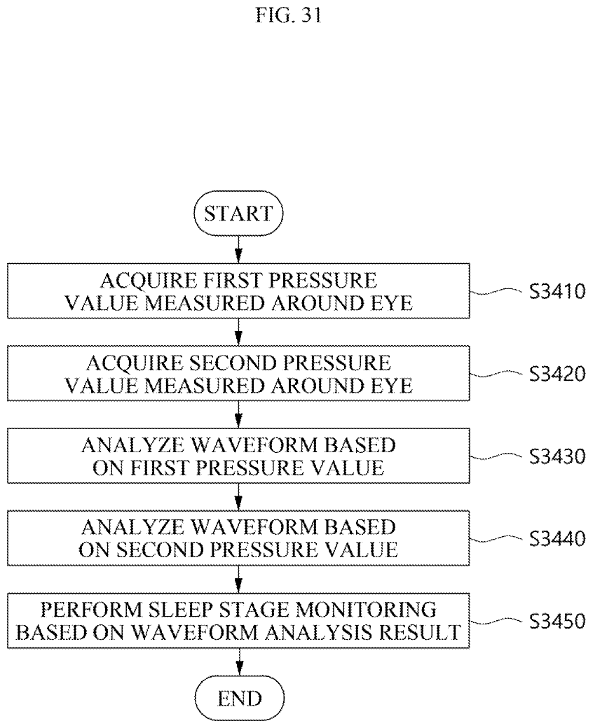

FIG. 31 is a diagram for illustratively describing a method for determining the movement of eye and a method for monitoring the sleep stage by a bio-signal measuring device according to another embodiment.

Referring to FIG. 31, the bio-signal measuring device 1000 according to another embodiment may perform a step of acquiring a first pressure value measured around the eye (S3410), a step of acquiring a second pressure value measured around the eye (S3420), a step of analyzing a waveform based on the first pressure value (S3430), a step of analyzing the waveform based on the second pressure value (S3440) and a step of monitoring the sleep stage based on the waveform analysis result (S3450).

The first pressure value may be a pressure value measured in the upper side or lower side of the eye, and the second pressure value may be a pressure value measured in the left side or right side of the eye.

When the bio-signal measuring device 1000 monitors the sleep stage based on the first pressure value and the second pressure value, a more accurate result may be acquired compared to monitoring the sleep stage based on either the first pressure value or the second pressure value.

Since the method in which the bio-signal measuring device 1000 generates the waveform, determines the movement of eye, and monitors the sleep stage based on the pressure value is the same as or corresponds to that described above with reference to FIGS. 28 to 30, duplicate description thereof will be omitted.

Meanwhile, for convenience of description, it has been described that a series of operations of generating the waveform, determining the movement of eye, and monitoring the sleep stage based on the pressure value around the eye measured from the bio-signal measuring device 1000 are performed by the bio-signal measuring device 1000, but is not limited thereto. The series of operations of generating the waveform, determining the movement of eye, and monitoring the sleep stage based on the pressure value around the eye measured from the bio-signal measuring device 1000 may also be performed by the server 2000 or the user terminal 3000.

5. Bio-Signal Analysis Algorithm

As described above, the bio-signal measuring device 1000 according to an embodiment may acquire the bio-signal generated from the human body during sleep and then extract various indicators based on the acquired bio-signal.

According to one embodiment, the bio-signal measuring device 1000 may acquire the ballistocardiogram signal generated from the human body during sleep, and extract at least one indicator based on the acquired ballistocardiogram signal.

The ballistocardiogram signal acquired by the bio-signal measuring device 1000 is the bio-signal generated during sleep, and is the bio-signal acquired cumulatively over a long period of time. Accordingly, waveforms created from the ballistocardiogram signal acquired by the bio-signal measuring device 1000 may be formed differently for each person even when sleeping in the same posture, and may be formed differently depending on the sleeping posture even in the case of the same person.

Due to the characteristics of the ballistocardiogram signal acquired during sleep described above, a simple algorithm has limitations in extracting indicators and monitoring the sleep stage through the analysis of the ballistocardiogram signal acquired during sleep, and thus a pre-trained neural network model specifically trained for this purpose should be used.

The neural network model for analyzing the ballistocardiogram signal may be stored in the server 2000, and the server 2000 may receive the bio-signal from the bio-signal measuring device 1000 and extract at least one indicator using the neural network model. Alternatively, the neural network model for analyzing the ballistocardiogram signal may be stored in the bio-signal measuring device 1000. In this case, the bio-signal measuring device 1000 may extract at least one indicator from the ballistocardiogram signal using the stored neural network model.

In order for the bio-signal measuring device 1000 to store the neural network model in the memory and analyze the ballistocardiogram signal using the neural network model, the structure of the neural network model needs to be light. Accordingly, there is a need to develop a neural network model capable of having a lightweight structure so that it can be stored in the device 1000 and still achieve high accuracy.

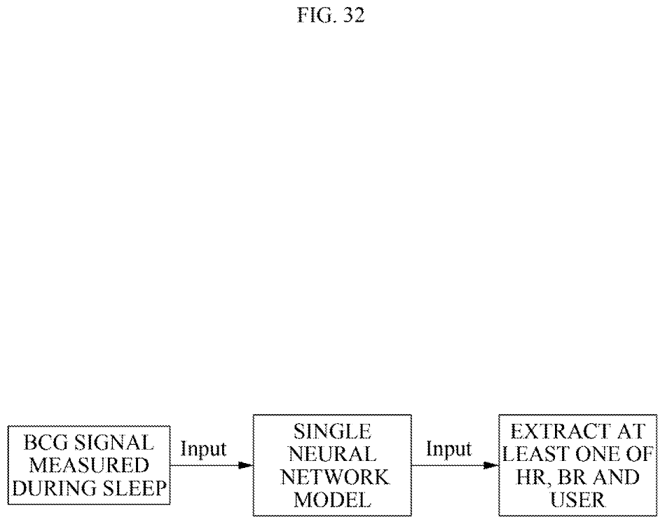

FIGS. 32 to 34 are diagrams for illustratively describing the neural network model operable in the bio-signal measuring device according to an embodiment.

Referring to FIG. 32, the bio-signal measuring device 1000 according to an embodiment may include a single neural network model.

The neural network model may receive the ballistocardiogram signal to extract a plurality of indicators. The neural network model may receive the ballistocardiogram signal to extract a first indicator and a second indicator. The first indicator and the second indicator may be extracted based on the ballistocardiogram signal, but may be extracted through a single neural network model. The first indicator may be at least one of heart rate, breathing rate, entropy, breathing amplitude, and movement of eye, and the second indicator may be an indicator different from the first indicator.

The neural network model may perform a function of extracting the first indicator and the second indicator from the ballistocardiogram signal, and may be trained using first training data and second training data. The first training data may include data related to the first indicator and first labeling data. The second learning data may include data related to the second indicator and second labeling data. More specifically, the neural network model may acquire a first output value related to the first indicator and a second output value related to the second indicator after receiving the ballistocardiogram signal. Thereafter, the neural network model may be trained by updating the neural network model based on the error value calculated by considering the difference between the first output value and the first labeling data and the difference between the second output value and the second labeling data.

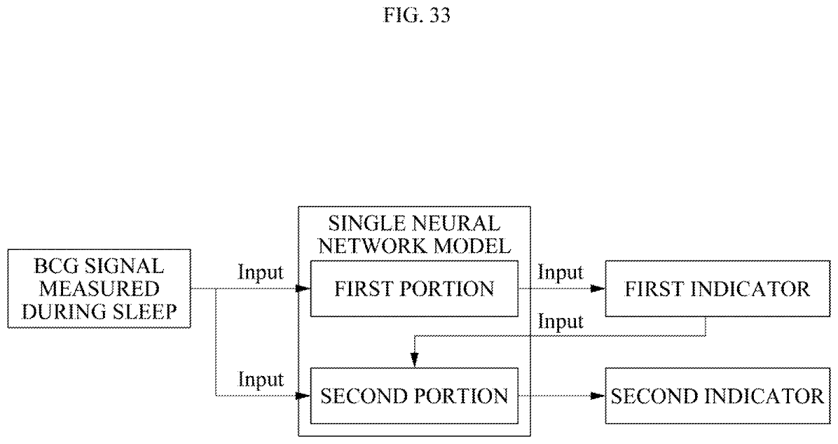

Referring to FIG. 33, the neural network model may be physically or logically separated within one model. A first part of the neural network model may be trained to output the first indicator from the ballistocardiogram signal, and a second part may be trained to output a second indicator based on the ballistocardiogram signal and the first indicator.

The second part of the neural network model may be trained to output the second indicator from the ballistocardiogram signal, and may be trained to output the second indicator using the first indicator as additional input data.

The first indicator and the second indicator extracted from the neural network model may be correlated with each other. The neural network model can simultaneously output the first indicator and the second indicator with high accuracy despite using one neural network model by considering the correlation.

As a more specific example, the neural network model may be trained to acquire heart rate and breathing rate from the ballistocardiogram signal. The neural network model may be trained using first training data related to heart rate and second training data related to breathing rate.

The neural network model may include a first part trained to acquire heart rate from the ballistocardiogram signal, and may include a second part trained to acquire breathing rate from the ballistocardiogram signal. The second part of the neural network model may be trained to acquire breathing rate from the ballistocardiogram signal, and may be trained to acquire breathing rate by additionally considering heart rate output from the first part.

The ballistocardiogram signal may be a ballistocardiogram signal generated by the user during sleep and measured by the bio-signal measuring device 1000. The ballistocardiogram signal may be the ballistocardiogram signal measured for a predetermined time by the bio-signal measuring device 1000. The ballistocardiogram signal may be the ballistocardiogram signal continuously measured by the bio-signal measuring device 1000. The ballistocardiogram signal may be a plurality of types of ballistocardiogram signals measured by the bio-signal measuring device 1000. The plurality of types may include at least one of a first type, a second type, and a third type determined based on the movement of the user.

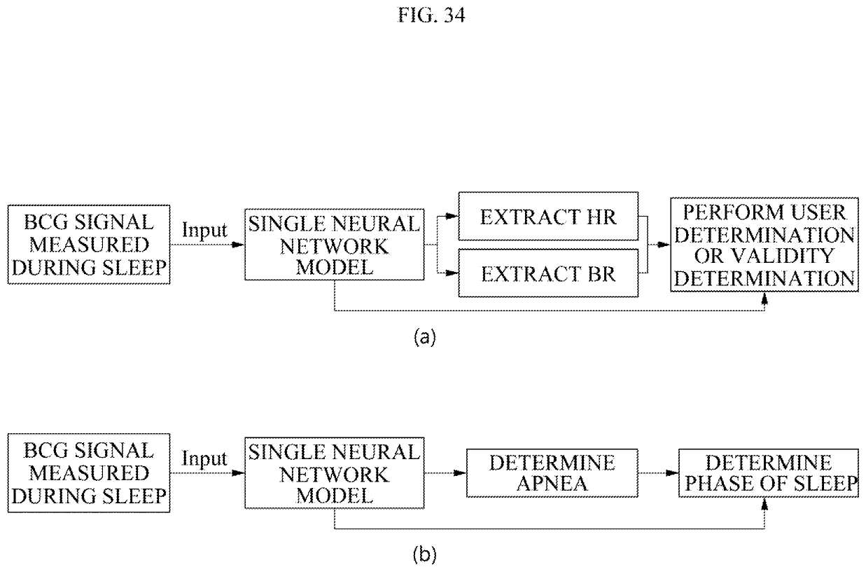

Referring to (a) of FIG. 34, the bio-signal measuring device 1000 according to an embodiment may receive the ballistocardiogram signal measured during sleep and output the first indicator and the second indicator using a single neural network model, and may perform user determination or validity determination based on the first indicator and the second indicator.

The user determination and validity determination may be performed the same as or corresponding to the method for determining whether the user is located, the method for verifying whether the bio-signal is valid, and the method for determining whether the placement of the bio-signal measuring device is appropriate, as described with reference to FIGS. 11 and 12. Since the description related to this have been described above, duplicate description thereof will be omitted.

The bio-signal measuring device 1000 may simultaneously perform the user determination and the validity determination based on the ballistocardiogram signal using one neural network model. More specifically, since the user determination and validity determination are made based on heart rate and breathing rate extracted from the ballistocardiogram signal, the bio-signal measuring device 1000 may simultaneously perform the user determination and the validity determination through the single neural network model.

Referring to (b) of FIG. 34, the bio-signal measuring device 1000 according to an embodiment may receive the ballistocardiogram signal measured during sleep and determine apnea and the phase of sleep using the single neural network model.

The bio-signal measuring device 1000 may determine apnea based on the ballistocardiogram signal measured during sleep using the neural network model. The bio-signal measuring device 1000 determines the phase of sleep based on the ballistocardiogram signal measured during sleep, but may determine the phase of sleep by additionally considering the apnea determination result.

For example, the bio-signal measuring device 1000 may determine apnea based on the ballistocardiogram signal through the first part of the neural network model, and may determine the phase of sleep based on the ballistocardiogram signal and the apnea determination result through the second part of the neural network model.

6. Sleep Stage Monitoring Through Bio-Signal Analysis and Method for Providing Alarm

The bio-signal measuring device 1000 according to an embodiment may perform the sleep monitoring based on at least one bio-signal measured during sleep. The sleep monitoring may be to continuously determine the phase of sleep.

The phases of sleep can be divided into the rapid movement of eye (REM) sleep phase and the non-REM sleep phase, and the non-REM sleep can be divided into a plurality of phases. In general, the REM sleep phase and a non-REM sleep phase may alternate multiple times during sleep based on duration, and different conditions may be provided depending on which phase of sleep the user wakes up from.

As such, it is important to determine the phase of sleep of the user so that the user can wake up with the optimal condition. When the phase of sleep of the user is accurately determined, an alarm may be provided so that the user wakes up at the most appropriate timing.

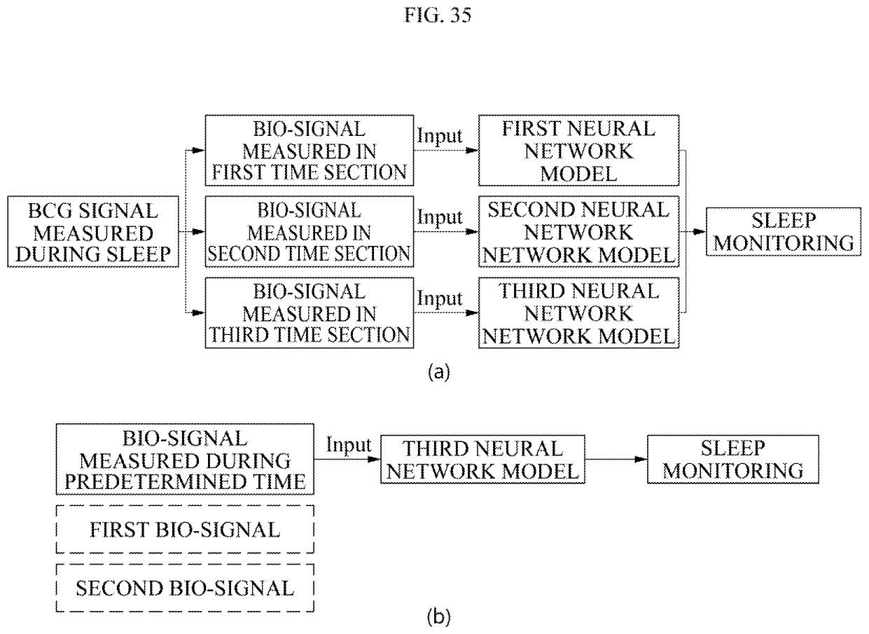

FIG. 35 is a diagram for describing a detailed method for monitoring the sleep stage using a pre-trained neural network model by the bio-signal measuring device according to an embodiment.

Referring to (a) of FIG. 35, the bio-signal measuring device 1000 may acquire the bio-signal (e.g., ballistocardiogram signal) measured during sleep. The ballistocardiogram signal is a signal acquired cumulatively during sleep, and may include a first ballistocardiogram signal measured in a first time period, a second ballistocardiogram signal measured in a second time period, and a third ballistocardiogram signal measured in a third time period. The first time period to third time period may be time periods having different lengths.

More specifically, assuming that the user sleeps for n hours, he/she may sleep for x hours first, wakes up for y hours in the middle, and then sleeps for z hours again. The z hours may be the first time period, and the first ballistocardiogram signal may be a ballistocardiogram signal measured during the x hours. The z hours may be a second time period, and the second ballistocardiogram signal may be a ballistocardiogram signal measured during z hours. In this case, the x hours and the z hours may have different lengths.

The bio-signal measuring device 1000 may include a first neural network model trained to extract an indicator for monitoring the sleep stage from the first ballistocardiogram signal measured in the first time period, a second neural network model trained to extract an indicator for monitoring the sleep stage from the second ballistocardiogram signal measured in the second time period, and a third neural network model trained to extract an indicator for monitoring the sleep stage from the third ballistocardiogram signal measured in the third time period.

The first neural network model may be trained to extract the indicator for monitoring the sleep stage based on the ballistocardiogram signal acquired during the first time period, the second neural network model may be trained to extract the indicator for monitoring the sleep stage based on the ballistocardiogram signal acquired during the second time period, and the third neural network model may be trained to extract the indicator for monitoring the sleep stage based on the ballistocardiogram signal acquired during the third time period.

Since the first to third neural network models are trained by targeting the ballistocardiogram signals acquired during different time periods, more accurate monitoring of the sleep stage is possible.

More specifically, the first neural network model may be trained to monitor the sleep stage from the ballistocardiogram signal acquired for a relatively short time period (e.g., for 2 hours), the second neural network model may be trained to monitor the sleep stage from the ballistocardiogram signal acquired for a medium time period (e.g., for 4 hours), and the third neural network model may be trained to monitor the sleep stage from the ballistocardiogram signal acquired for a relatively long time period (e.g., for 10 hours).

Referring to (b) of FIG. 35, the bio-signal measuring device 1000 may acquire the bio-signal (e.g. ballistocardiogram signal) measured for a predetermined time period. The predetermined time period may include a time period during which the user is sleeping and a time period during which the user is briefly awake while sleeping.

For example, the ballistocardiogram signal measured for the predetermined time period may include the first ballistocardiogram signal measured in the first time period, the second ballistocardiogram signal measured in the second time period, and the third ballistocardiogram signal measured in the third time period. In this case, at least one of the first ballistocardiogram signal to third ballistocardiogram signal may be the ballistocardiogram signal measured in a wake state. For example, the first ballistocardiogram signal and the third ballistocardiogram signal may be ballistocardiogram signals measured during sleep, and the second ballistocardiogram signal may be the ballistocardiogram signal measured in the wake state.

The bio-signal measuring device 1000 may monitor the sleep stage based on the ballistocardiogram signal measured for the predetermined time period using a pre-trained neural network model. The bio-signal measuring device 1000 may monitor the sleep stage based on the first ballistocardiogram signal measured during sleep during the predetermined time period and the second ballistocardiogram signal measured in the wake state using a pre-trained neural network model.

In other words, the bio-signal measuring device 1000 can accurately monitor the sleep stage using a pre-trained neural network model even when the ballistocardiogram signal (e.g., noisy data) measured while the user is briefly awake is included in the ballistocardiogram signal measured for the predetermined time period.

The neural network model may be trained to extract the indicator for monitoring the sleep stage from the ballistocardiogram signal using the first ballistocardiogram signal measured during sleep and noise data (e.g., the second ballistocardiogram signal measured in a wake state) as training data.

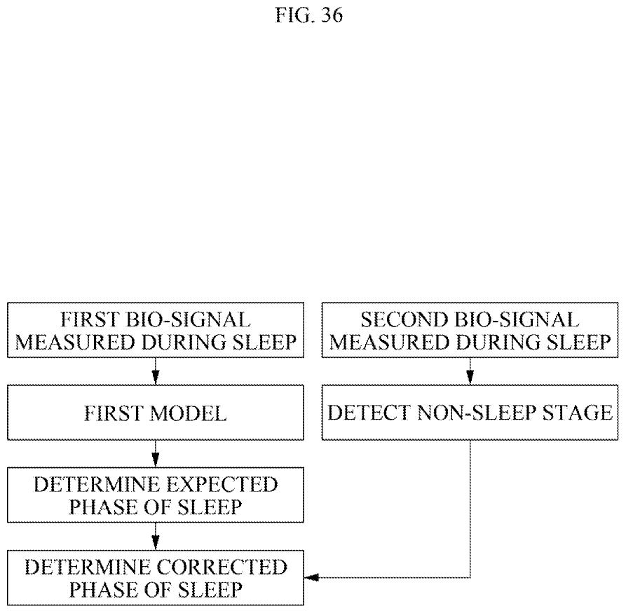

FIGS. 36 to 38 are diagrams for describing a method for correcting a predicted sleep stage using an additional indicator by the bio-signal measuring device according to an embodiment.

Referring to FIG. 36, the bio-signal measuring device 1000 according to an embodiment may generate an expected phase of sleep based on the first bio-signal (e.g. ballistocardiogram signal) measured during sleep through a first model. Since generating the expected phase of sleep using the first model by the bio-signal measuring device 1000 is the same as or corresponds to the content described above with reference to FIG. 35, duplicate description thereof will be omitted.

The bio-signal measuring device 1000 may detect a non-sleep stage through a second bio-signal measured during sleep. For example, the second bio-signal may be the same as the first bio-signal. As another example, the second bio-signal may be different from the first bio-signal.

The bio-signal measuring device 1000 may determine at least one of the movement of the user, presence and absence of the user, the movement of eye of the user, an activity amount, and entropy using the ballistocardiogram signal or the sound signal. The bio-signal measuring device 1000 may determine whether or not the user is in the non-sleep stage based on at least one of the movement of the user, the presence and absence of the user, the movement of eye of the user, the activity amount, and entropy using the ballistocardiogram signal or the sound signal.

The bio-signal measuring device 1000 may generate a corrected phase of sleep by correcting the expected phase of sleep generated through the first model using the non-sleep stage detection result of the user as an indicator.

Illustratively, the bio-signal measuring device 1000 may determine the expected phase of sleep using the first model, and the expected phase of sleep may be that the user is in a first phase of sleep in the first time period, a second phase of sleep in the second time period, and a third phase of sleep in the third time period.

The bio-signal measuring device 1000 may detect the non-sleep stage based on the second bio-signal. When it is determined that the user is in the non-sleep stage as the non-sleep stage detection result, the bio-signal measuring device 1000 may correct the expected phase of sleep by correcting that the user is in the non-sleep stage in the second time period.

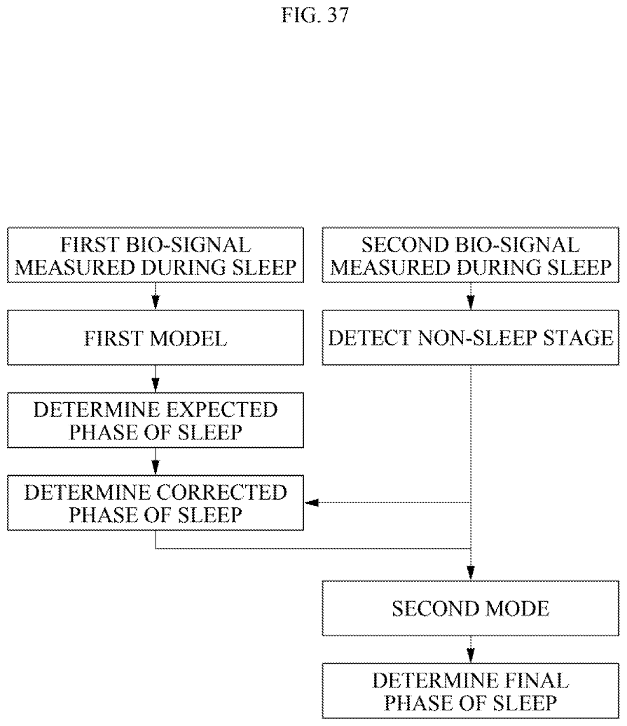

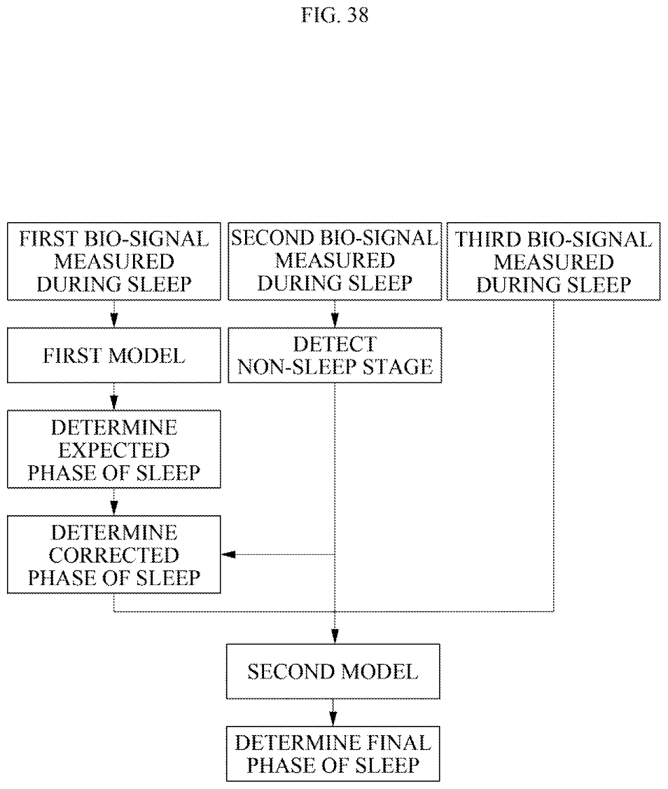

Referring to FIGS. 37 and 38, the bio-signal measuring device 1000 according to an embodiment may additionally use a second model to determine a final phase of sleep.

The bio-signal measuring device 1000 may determine the final phase of sleep using the result of the corrected final phase of sleep determination determined by the method described above through the second model and the result of the non-sleep stage determination determined based on the second bio-signal as input data.

Alternatively, unlike the drawing, the bio-signal measuring device 1000 may determine the phase of sleep using the result of the corrected phase of sleep determination determined by the method described above through the second model as input data.

The bio-signal measuring device 1000 may determine the final phase of sleep by additionally considering a third bio-signal measured during sleep. The third bio-signal may be the same as or different from the first bio-signal and the second bio-signal.

The bio-signal measuring device 1000 may determine the final phase of sleep using at least one of the result of the corrected phase of sleep determination acquired through the first model, the non-sleep stage detection result, and the third bio-signal.

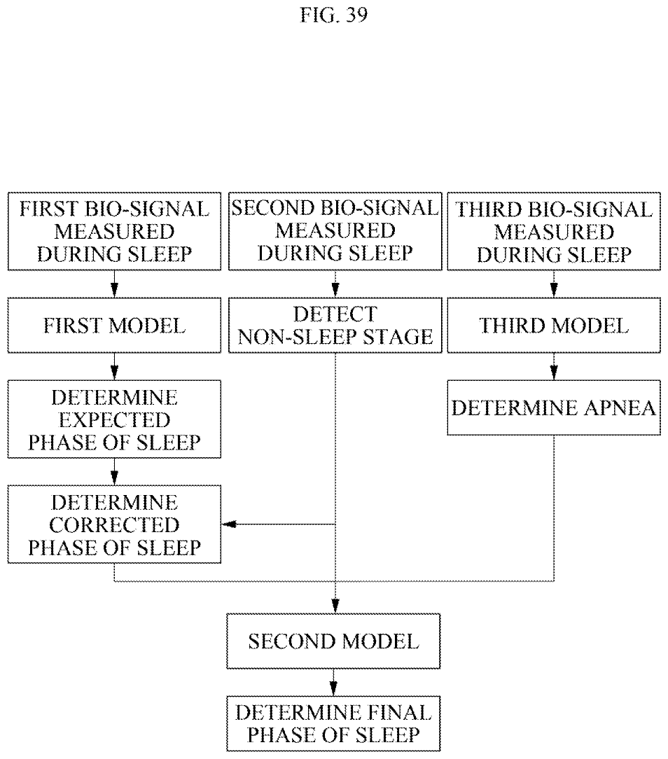

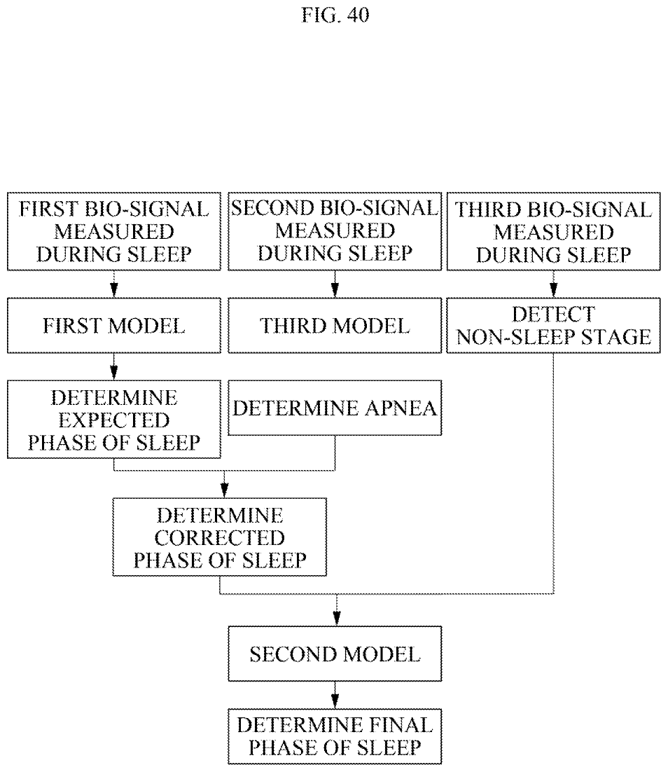

FIGS. 39 and 40 are diagrams for describing a method for correcting a predicted sleep stage using an additional indicator by a bio-signal measuring device according to another embodiment.

Referring to FIG. 39, the bio-signal measuring device 1000 may determine apnea using the third bio-signal measured during sleep as input data of the third model. Since the specific method for determining apnea by the bio-signal measuring device 1000 using the third model has been described above, duplicate description thereof will be omitted.

The bio-signal measuring device 1000 may use at least one of the result of corrected phase of sleep determination derived through the first model, the non-sleep stage detection result derived based on the second bio-signal, and the apnea determination result derived through the third model to determine the final phase of sleep. The bio-signal measuring device 1000, using the second model, may determine the final phase of sleep based on at least one of the result of corrected phase of sleep determination derived through the first model, the non-sleep stage detection result derived based on the second bio-signal, and the apnea determination result derived through the third model.

As a more specific example, the bio-signal measuring device 1000 may determine apnea through the third model. The apnea determination result may include determination results regarding an apnea period during sleep, intensity of apnea, sleeping posture when apnea occurs, and number and frequency of occurrence of apnea. The bio-signal measuring device 1000 may determine the phase of sleep by additionally considering the apnea determination result as described above.

Referring to FIG. 40, the bio-signal measuring device 1000 may determine apnea using the third model, and may generate a corrected phase of sleep by correcting the expected phase of sleep determined through the first model based on the apnea determination result.

The bio-signal measuring device 1000 may determine the expected phase of sleep of the user for each time period determined through the first model, and at the same time, determine apnea of the user for each time period. The bio-signal measuring device 1000 may generate the corrected phase of sleep by correcting the expected phase of sleep using the apnea determination result as an additional indicator.

The bio-signal measuring device 1000 may determine the final phase of sleep using at least one of the result of corrected phase of sleep determination corrected by the method described above and the non-sleep stage detection result determined based on the second bio-signal as input data of the second model.

7. Method for Providing Wake-Up Alarm Based on Sleep Monitoring

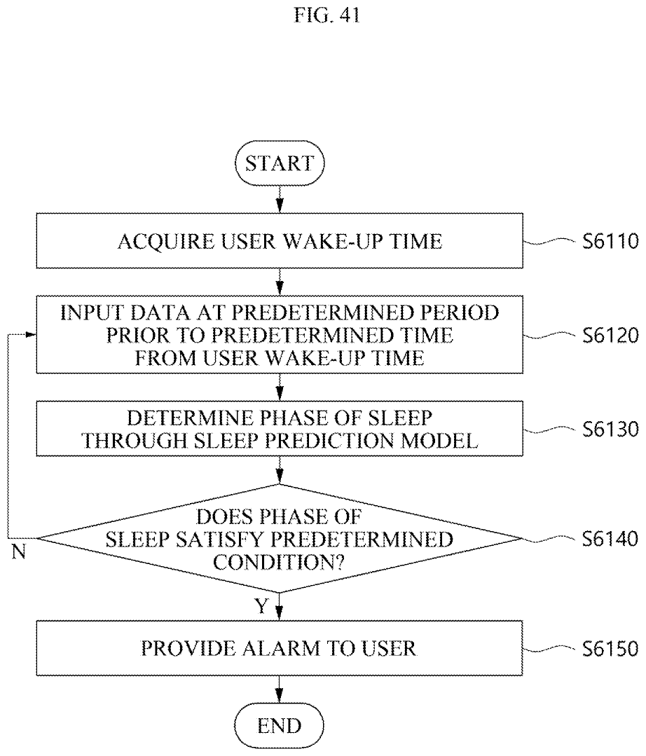

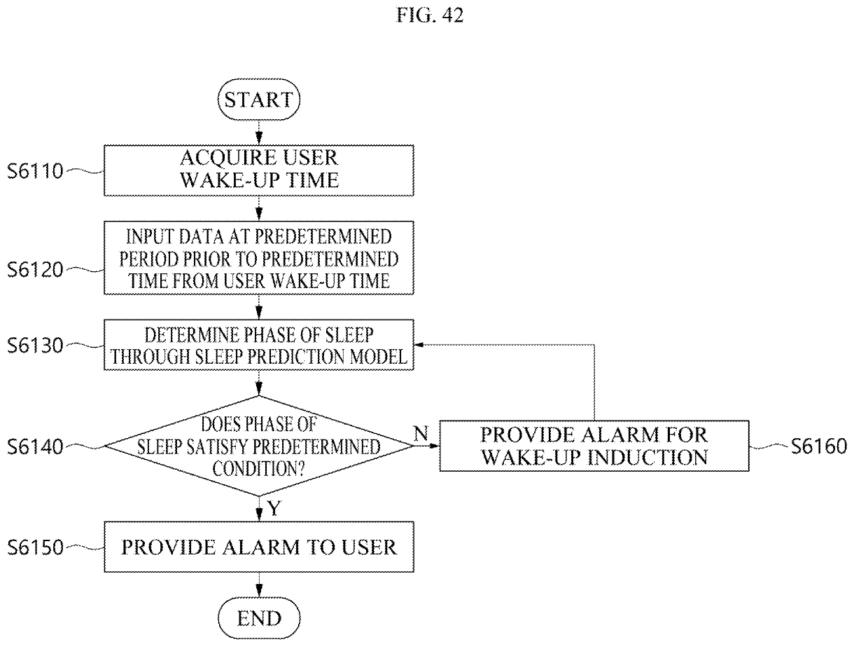

FIGS. 41 and 42 are diagrams for describing a method for providing an alarm to the user based on a sleep monitoring result by the bio-signal measuring device according to an embodiment.

Referring to FIGS. 41 and 42, the bio-signal measuring device 1000 may perform a step of acquiring user wake-up time (S6110), a step of inputting data at a predetermined period prior to a predetermined time from the user wake-up time (S6120), a step of determining a phase of sleep through a sleep prediction model (S6130), a step of determining whether the phase of sleep satisfies a predetermined condition (S6140), a step of providing an alarm for wake-up induction (S6160), and a step of providing the alarm to the user (S6150).

The bio-signal measuring device 1000 may acquire the wake-up time of the user from input of the user. Thereafter, the bio-signal measuring device 1000 may acquire data at a predetermined interval prior to a predetermined time from the acquired wake-up time of the user. In this case, the data may be at least one bio-signal measured during sleep or at least one indicator extracted based on the at least one bio-signal.

The predetermined time and predetermined period may be acquired from the input of the user. Illustratively, the predetermined time may be 30 minutes, and the predetermined time period may be 1 minute. In this case, the bio-signal measuring device 1000 may receive data at intervals of 1 minute 30 minutes before the wake-up time of the user, and determine the sleep stage based on this.

The bio-signal measuring device 1000 may determine the phase of sleep of the user using a sleep prediction model. Thereafter, the bio-signal measuring device 1000 may determine whether the phase of sleep of the user satisfies the predetermined condition. When it is determined that the phase of sleep of the user satisfies the predetermined condition, the bio-signal measuring device 1000 may provide the alarm so that the user wakes up.

The predetermined condition may be related to whether the phase of sleep of the user corresponds to a predetermined phase of sleep. For example, the phase of sleep of the user may include the REM sleep phase and the non-REM sleep phase. The non-REM sleep stage may include a deep sleep phase and a light sleep phase. In this case, the predetermined condition may be related to whether the phase of sleep of the user corresponds to the light sleep phase.

More specifically, since the user is deeply asleep in the deep sleep phase of the non-REM sleep phase, it is not desirable for the user to wake up in the deep sleep phase. In addition, since it can be considered that the user is relatively deeply asleep in the REM sleep phase, it is not desirable for the user to wake up in the REM sleep phase. Waking up from the light sleep phase in which the user is lightly asleep is most preferable. In this case, since a better condition can be maintained, the bio-signal measuring device 1000 can provide the alarm so that the user can wake up when the sleep stage of the user is in the light sleep phase, after determining the phase of sleep of the user.

When it is determined that the phase of sleep of the user does not satisfy the predetermined condition, the bio-signal measuring device 1000 may provide an alarm for inducing wake up to the user. When it is determined that the current time is close to the wake-up time of the user and the sleep stage of the user does not satisfy the predetermined condition, the bio-signal measuring device 1000 may separately provide an alarm for inducing wake-up so that the user can wake up slowly.

When the phase of sleep does not satisfy the predetermined condition, the bio-signal measuring device 1000 may provide an alarm to the user so that the phase of sleep of the user becomes a predetermined phase of sleep (e.g., the light sleep phase). In this case, the alarm may be a vibration alarm, and the bio-signal measuring device 1000 may provide the alarm to the user by adjusting the intensity of vibration.

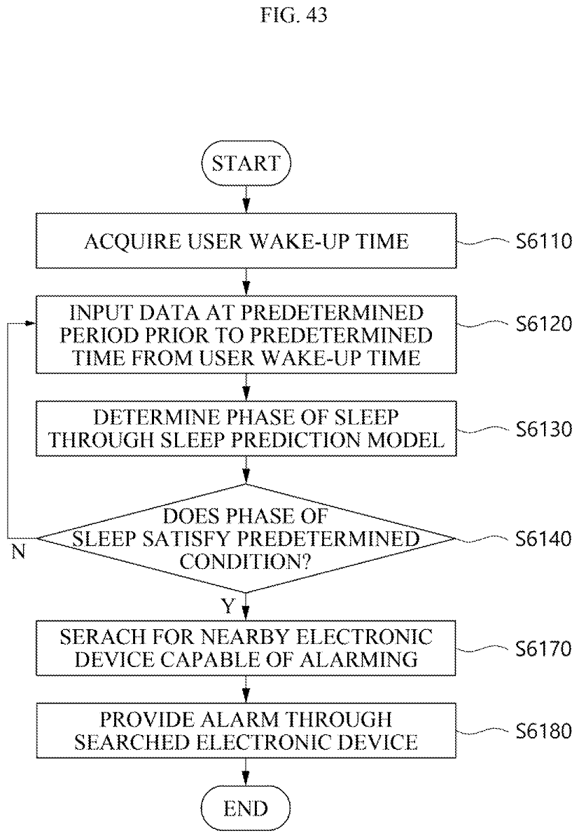

FIG. 43 is a diagram for describing a method for providing the alarm to the user through a peripheral device based on the sleep monitoring result by the bio-signal measuring device according to an embodiment.

Referring to FIG. 43, the bio-signal measuring device 1000 may additionally perform a step of searching for a nearby electronic device capable of alarming (S6170) and a step of providing an alarm through the searched electronic device (S6180).

The bio-signal measuring device 1000 may determine whether the phase of sleep of the user satisfies the predetermined condition by the method described above through FIG. 42, and may search for a nearby electronic device capable of alarming when it is determined that the phase of sleep of the user satisfies the predetermined condition. The electronic device may be a user terminal, a speaker, a television, or other household appliances.

When it is determined that the phase of sleep of the user satisfies the predetermined condition, the bio-signal measuring device 1000 may provide an alarm to the user through at least one of a plurality of nearby electronic devices capable of alarming.

Illustratively, when it is determined that the phase of sleep of the user satisfies a predetermined condition, the bio-signal measuring device 1000 may output sound through an external speaker communicatively connected to the bio-signal measuring device 1000. As another example, when it is determined that the phase of sleep of the user satisfies the predetermined condition, the bio-signal measuring device 1000 may output light through a lamp communicatively connected to the bio-signal measuring device 1000.

8. Method for Providing Sleep Stage Monitoring Result

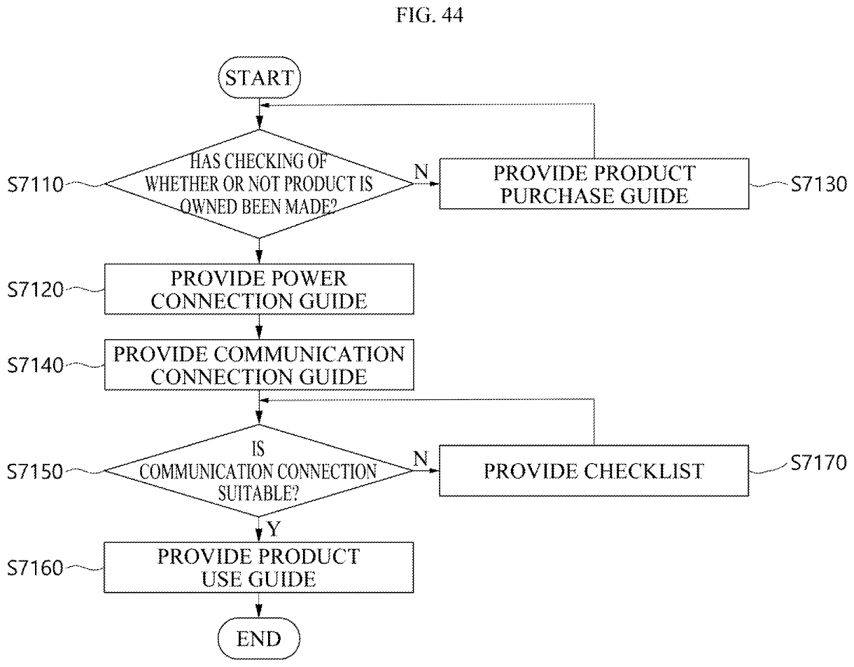

FIGS. 44 to 47 are diagrams for illustratively describing a method for a user terminal to guide a user on how to use the bio-signal measuring device according to an embodiment.

Referring to FIG. 44, the user terminal 3000 may perform a step of checking whether or not the product is owned by the user (S7110), a step of providing a power connection guide (S7120), a step of providing a product purchase guide (S7130), a step of providing a communication connection guide (S7140), a step of determining whether the communication connection is appropriate (S7150), a step of providing a product user guide (S7160), and a step of providing a checklist (S7170).

The user terminal 3000 may output an interface for asking the user to check whether he/she owns the bio-signal measuring device 1000 through a display.

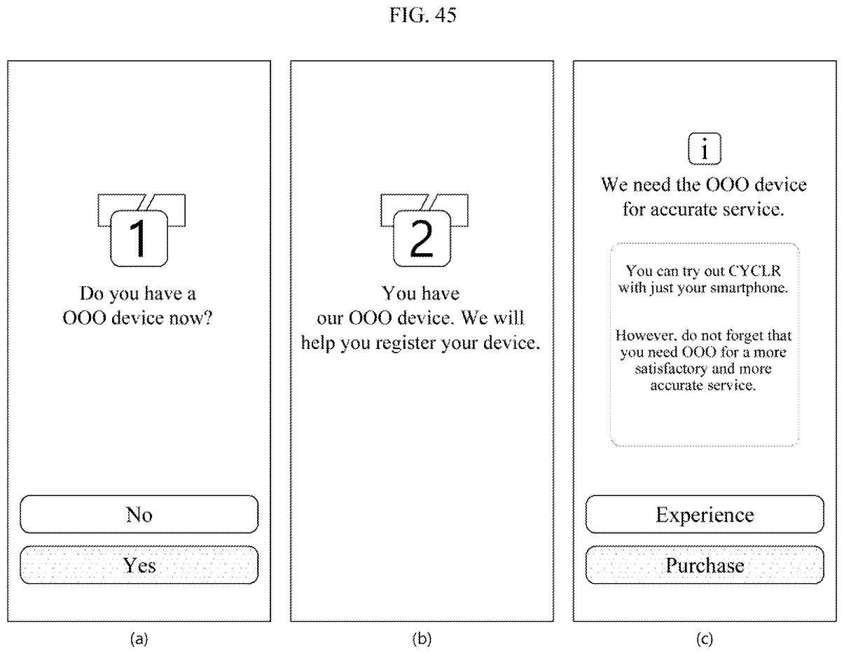

For example, as illustrated in (a) of FIG. 45, the user terminal 3000 may output at least one object capable of receiving a response of the user as to whether or not the user owns the bio-signal measuring device 1000 to the user through a display.

The user terminal 3000 may determine whether or not the user owns the bio-signal measuring device 1000 based on the user input. When it is determined that the user owns the bio-signal measuring device 1000, the user terminal 3000 may provide the power connection guide for the bio-signal measuring device 1000 to the user. When it is determined that the user does not own the bio-signal measuring device 1000, the user terminal 3000 may provide a guide on how to purchase the bio-signal measuring device 1000.

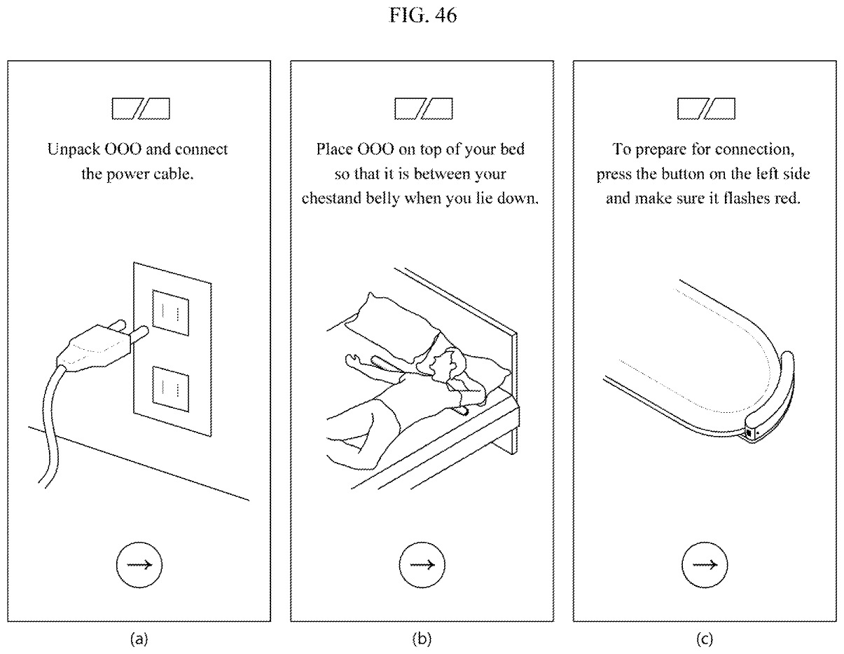

For example, as illustrated in (b) of FIG. 45, when it is determined that the user owns the bio-signal measuring device 1000, the user terminal 3000 may output a guide on how to register the device or a power connection guide to the user through a display. In addition, when it is determined that that the user does not own the bio-signal measuring device 1000, the user terminal 3000 may output a message related to a method for purchasing the bio-signal measuring device 1000 through a display as illustrated in (c) of FIG. 45 or a method for experiencing the device as illustrated in (a) of FIG. 46.

The user terminal 3000 may output an interface for guiding a communication connection with the bio-signal measuring device 1000 to the user through a display. Thereafter, the user terminal 3000 may output a user guide of the bio-signal measuring device 1000 when it is determined that the communication connection with the bio-signal measuring device 1000 is appropriate, and may output a checklist for communication connection with the bio-signal measuring device 1000 when it is determined that the communication connection with the bio-signal measuring device 1000 is not appropriate.

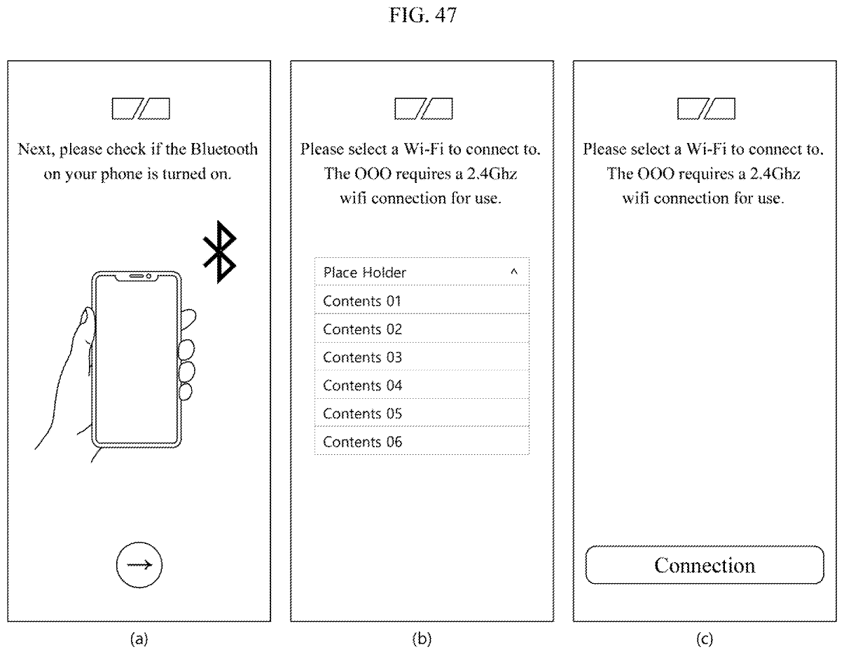

For example, the user terminal 3000 may output a screen guiding the user on how to use the bio-signal measuring device 1000 through a display as illustrated in (b) and (c) of FIG. 46, and output a screen guiding the communication connection with the bio-signal measuring device 1000 through a display as illustrated in (a) to (c) of FIG. 47.

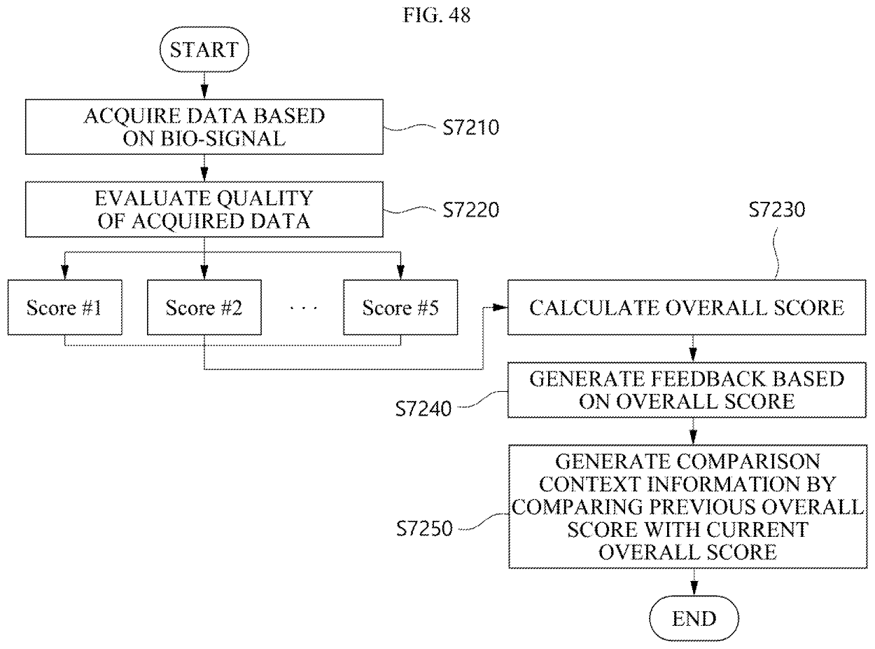

FIGS. 48 to 52 are diagrams for illustratively describing a method for outputting a sleep stage monitoring to the user by the user terminal according to an embodiment.

Referring to FIG. 48, the user terminal 3000 may perform a step of acquiring data based on the bio-signal (S7210), a step of evaluating quality of the data (S7220), a step of calculating an overall score (S7230), a step of generating feedback based on the overall score (S7240), and a step of generating comparison context information by comparing the previous overall score with the current overall score (S7250).

The user terminal 3000 may acquire at least one bio-signal generated during sleep through the bio-signal measuring device 1000, and may perform the analysis of the at least one bio-signal through a pre-trained neural network model. Since the method in which the user terminal 3000 performs the analysis of at least one bio-signal has been described above, duplicate description thereof will be omitted.

The user terminal 3000 may calculate at least one score through the analysis of at least one bio-signal. The user terminal 3000 may determine a first sleep score, a second sleep score, a third sleep score, a fourth sleep score, and a fifth sleep score based on the result of the analysis of at least one bio-signal.

The user terminal 3000 may decide the first sleep score related to deep sleep through the analysis of at least one bio-signal. The user terminal 3000 may extract a first indicator related to sleep duration and extract a second indicator related to deep sleep duration through the analysis of at least one bio-signal. The user terminal 3000 may decide the first sleep score related to the deep sleep based on the first indicator and the second indicator.

The user terminal 3000 may decide the first sleep score based on a ratio between the deep sleep duration and the sleep duration. The user terminal 3000 may decide the first sleep score by assigning a weight to the ratio between the deep sleep duration and the sleep duration.

For example, the user terminal 3000 may determine decide the first sleep score by assigning a first weight thereto when the ratio between the deep sleep duration and the sleep duration is within a first range. The user terminal 3000 may decide the first sleep score by assigning a second weight thereto when the ratio between the deep sleep duration and the sleep duration is within a second range. The user terminal 3000 may decide the first sleep score by assigning a third weight thereto when the ratio between the deep sleep duration and the sleep duration is within a third range. The user terminal 3000 may decide the first sleep score by assigning a fourth weight thereto when the ratio between the deep sleep duration and the sleep duration is within a fourth range.

As a more specific example, referring to Table 1 below, the first sleep score may be decided by reflecting the first weight to the ratio between the deep sleep duration and the sleep duration when the ratio between the deep sleep duration and the sleep duration exceeds y and is less than or equal to z. The first sleep score may be decided by reflecting the second weight to the ratio between the deep sleep duration and the sleep duration when the ratio between the deep sleep duration and the sleep duration exceeds x and is less than or equal to y. The first sleep score may be decided by reflecting the third weight to the ratio between the deep sleep duration and the sleep duration when the ratio between the deep sleep duration and the sleep duration exceeds z. The first sleep score may be decided by reflecting the fourth weight and the fifth weight to the ratio between the deep sleep duration and the sleep duration the ratio between the deep sleep duration and the sleep duration is x or less.

TABLE 1

$First score = ( first weight ) \cdot if ( y < \frac{deep duration}{sleep duration} \leq z )$
$First score = ( second weight ) \cdot if ( x < \frac{deep duration}{sleep duration} \leq y )$
$First score = ( third weight ) \cdot if ( z > \frac{deep duration}{sleep duration} )$
$\begin{matrix}First score = ( forth weight ) \cdot \\ \frac{deep duration}{sleep duration} \cdot ( fifth weight ) \cdot if ( \frac{deep duration}{sleep duration} \leq x )\end{matrix}$

The user terminal 3000 may decide the second sleep score related to sleep efficiency through the analysis of at least one bio-signal. The user terminal 3000 may decide total duration of the user and sleep duration through the analysis of at least one bio-signal, and determine the second sleep score based on the total duration and the sleep duration.

The total duration may mean the total time from a first time point when the user starts to sleep to a second time point when the user wakes up. The total duration may be the time between the first time point and the second time point, and may include the time during which the non-sleep stage is continued when the user enters the non-sleep stage in the middle.

The sleep duration may be the time acquired by excluding the time during which the user is in non-sleep stage from the total duration. The sleep duration may mean only the time during which the sleep stage of the user is actually continued by excluding the time during which the non-sleep stage of the user from the total duration.

Illustratively, referring to Table 2 below, the second sleep score may be decided) by reflecting the first weight to a ratio between the sleep duration and the total duration.

TABLE 2
$Second score = ( first weight ) \cdot ( \frac{sleep duration}{total duration} )$

The user terminal 3000 may decide the third sleep score related to sleep latency through the analysis of at least one bio-signal. The user terminal 3000 may decide the sleep latency time from the time when the user starts preparing for sleep to the time when the user enters the phase of sleep through the analysis of at least one bio-signal, and may decide the third sleep score based on the sleep latency time.

The user terminal 3000 may determine a first time point when the user starts preparing for sleep and may determine a second time point when the user enters the sleep stage through the analysis of at least one bio-signal. The user terminal 3000 may decide the sleep latency time (e.g., the time between the first time point and the second time point) based on the first time point and second time point, and decide the third sleep score based on the sleep latency time.

The user terminal 3000 may decide the third sleep score by assigning a first weight thereto when the sleep latency time is within a first range. The user terminal 3000 may decide the third sleep score by assigning a second weight thereto when the sleep latency time is within a second range. The user terminal 3000 may decide the third sleep score by assigning a third weight thereto when the sleep latency time is within a third range.

As a more specific example, referring to Table 3 below, the third sleep score may be decided by reflecting the first weight to the sleep latency time when the sleep latency time exceeds 0 minutes and is less than or equal to x minutes. The third sleep score may be decided by reflecting the second weight to the sleep latency time when the sleep latency time exceeds x minutes and is less than or equal to y minutes.

TABLE 3

Third score = (first weight) · if 0 < sleep latency ≤ x
Third score = (second weight) · if (x < sleep latency ≤ y)

The user terminal 3000 may decide the fourth sleep score related to a wake condition through the analysis of at least one bio-signal. The user terminal 3000 may repeatedly determine the phase of sleep of the user a predetermined number of times during a predetermined time period through the analysis of at least one bio-signal.

The user terminal 3000 may repeatedly decide the phase of sleep of the user the predetermined number of times during the predetermined time period, and may decide the fourth sleep score related to the wake condition based on the number of times it is determined to be the first phase of sleep. In addition, the user terminal 3000 may repeatedly determine the phase of sleep of the user the predetermined number of times during the predetermined time period, and may decide the fourth sleep score related to the wake condition based on the number of times it is determined to be the first phase of sleep and the second phase of sleep.

The predetermined time period may be decided based on a target wake-up time point of the user. For example, when the target wake-up time point of the user is a first time point, the predetermined time period may be a time period of n minutes (e.g. 30 minutes) calculated backward from the first time point. The predetermined number of times may mean the number of times the predetermined time period is divided by any natural number. For example, when the predetermined time period is n minutes (e.g., 30 minutes), the predetermined number of times may be n times (e.g., 30 times).

As a more specific example, referring to Table 4 below, the fourth sleep score may be decided based on the number of times it is determined to be the first phase of sleep and the second phase of sleep by determining the phase of sleep of the user by the predetermined number of times (n times) during the predetermined time period.

TABLE 4

$ratio = \frac{number of ( first sleep stage ) + ( second sleep stage )}{n}$
Fourth score = 100 · ratio

The user terminal 3000 may determine the fifth sleep score related to the sleep duration through the analysis of at least one bio-signal. The user terminal 3000 may determine the first time point when the user enters a sleep stage and a second time point when the user wakes up through the analysis of at least one bio-signal. The user terminal 3000 may decide the sleep duration (e.g., the time between the first time point and the second time point) based on the first time point and the second time point, and decide the fifth sleep score based on the sleep duration.

The user terminal 3000 may decide the fifth sleep score by assigning a first weight thereto when the sleep duration is within a first range. The user terminal 3000 may decide the fifth sleep score by assigning a second weight thereto when the sleep duration is within a second range. The user terminal 3000 may decide the fifth sleep score by assigning a third weight thereto when the sleep duration is within a third range.

As a more specific example, referring to Table 5 below, the fifth sleep score may be decided by reflecting the first weight to the sleep duration when the sleep duration exceeds y hours and is less than or equal to z hours. The fifth sleep score may be decided by reflecting the second weight to the sleep duration when the sleep duration exceeds x hours and is less than or equal to y hours, or exceeds z hours. The fifth sleep score may be decided by reflecting the third weight to the sleep duration when the sleep duration is less than x hours.

TABLE 5

Fifth score = (first weight) · if (y < sleep duration ≤ z)
Fifth score = (second weight) · if(x < sleep duration ≤ y or
z < sleep duration)
Fifth score = (third weight) · if (sleep duration < x)

The user terminal 3000 may decide an overall score using at least one of the first sleep score to the fifth sleep score described above. The user terminal 3000 may decide the overall score by assigning the first weight to the first sleep score, assigning the second weight to the second sleep score, assigning the third weight to the third sleep score, and assigning the fourth weight to the fourth sleep score assigning the fifth weight to the fifth sleep score. The first weight to fifth weight may be determined to be the same or different from each other.

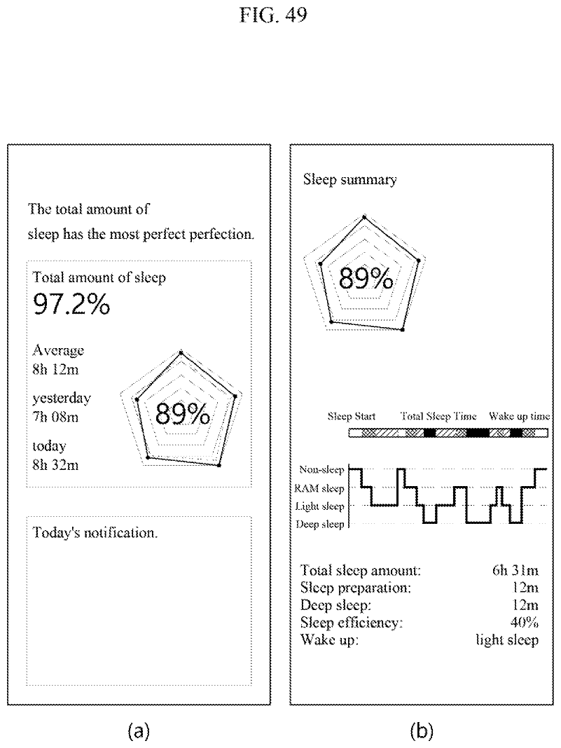

Referring to FIGS. 49 to 52, the user terminal 3000 may visually provide a sleep monitoring result based on the first sleep score to the fifth sleep score, which are decided by the method described above through a display, to the user.

The user terminal 3000 may provide the first sleep score to the fifth sleep score and the overall score to the user in the form of text, as illustrated in (a) of FIG. 49, and may visually provide the scores to the user through an arbitrary object. The user terminal 3000 may provide any one of the first sleep score to the fifth sleep score decided at the first time point to the user by comparing it with any one of the first sleep score to the fifth sleep score decided at the second time point. The first time point and the second time point may be different from each other.

The user terminal 3000 may output the first sleep score to the fifth sleep score in the form of a radar graph. For example, the user terminal 3000 may output a target object acquired by reflecting the first sleep score to the fifth sleep score on respective axes extending from the center to the vertex of a reference object (e.g. pentagonal object). The user terminal 3000 may output the overall score by overlapping it with the target object. In this case, the overall score may be decided based on the first sleep score to the fifth sleep score.

The user terminal 3000 may decide a total area of the target object as the overall score when at least one of the first sleep score to the fifth sleep score is lowered to a threshold value or less. For example, when the overall score is decided based on the first sleep score to the fifth sleep score, if at least one of the first sleep score to the fifth sleep score becomes significantly lower than the threshold value, the overall score may not correspond to the total area of the target object (e.g., the total area of the target object may be significantly lower than the overall score decided based on the first sleep score to the sixth sleep score). In this case, the user may feel a sense of difference between the overall score and the visual effect shown by the area of the target object and the overall score. Therefore, in this case, the user terminal 3000 may replace the overall score with the total area of the target object and output the same.

As illustrated in (b) of FIG. 49, the user terminal 3000 may visually provide the phase of sleep of the user determined during sleep to the user through a display. The user terminal 3000 may output an interface visually displayed differently according to the stages of sleep on a timeline. Accordingly, the user terminal 3000 can efficiently provide a state of change in the sleep stage of the user to the user on the timeline.

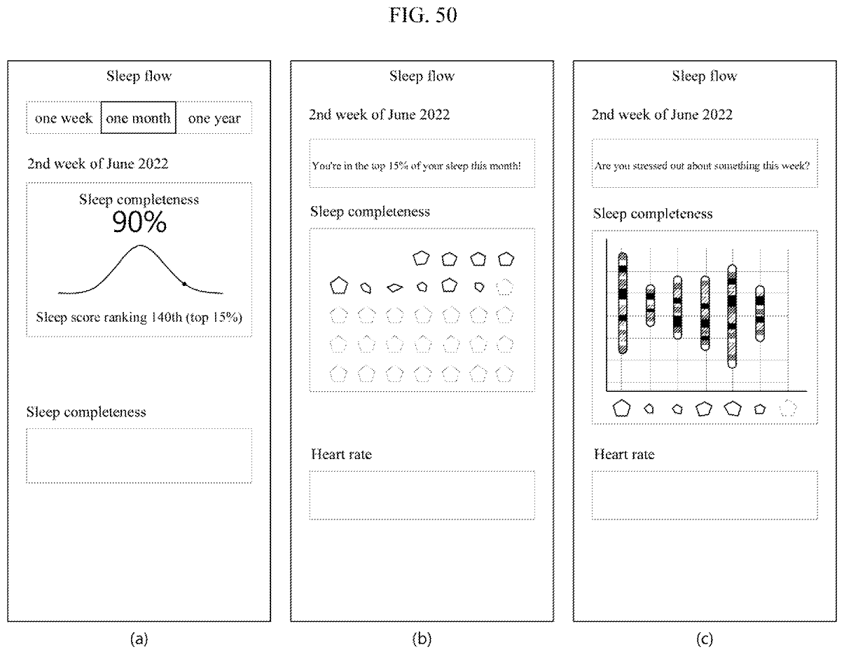

As illustrated in (a) of FIG. 50, the user terminal 3000 may provide a result of monitoring the sleep stage determined for a predetermined period of time (e.g., a week, a month, a year, etc.) to the user. The user terminal 3000 may provide a comparison score or a comparison rank with other users decided based on the first sleep score to the fifth sleep score to the user.

As illustrated in (b) of FIG. 50, the user terminal 3000 may display a plurality of objects corresponding to the predetermined period of time, and provide at least one of the first sleep score to the fifth sleep score to the user by sequentially mapping it to the plurality of objects. For example, the user terminal 3000 may generate a first object based on at least one of the first sleep score to the fifth sleep score decided based on the bio-signals measured on the first day, and generate a second object based on the at least one of the first sleep score to the fifth sleep score decided based on the bio-signals measured on the second day. Thereafter, the user terminal 3000 may display a plurality of reference objects by the number corresponding to one month, and provide the first object and the second object to the user by sequentially mapping the first and second objects to the reference objects.

As illustrated in (c) of FIG. 50, the user terminal 3000 may output a first interface displaying the phase of sleep of the user determined during sleep on the timeline and a second interface related to an object generated based on at least one of the first sleep score to the fifth sleep score through the display. The user terminal 3000 may display and output the first interface in a region adjacent to the second interface. By displaying and outputting the first interface and the second interface in the region adjacent to each other by the user terminal 3000, the user can easily grasp the change in his/her sleep stage on the timeline and at the same time, visually easily grasp the result of monitoring his/her sleep stage.

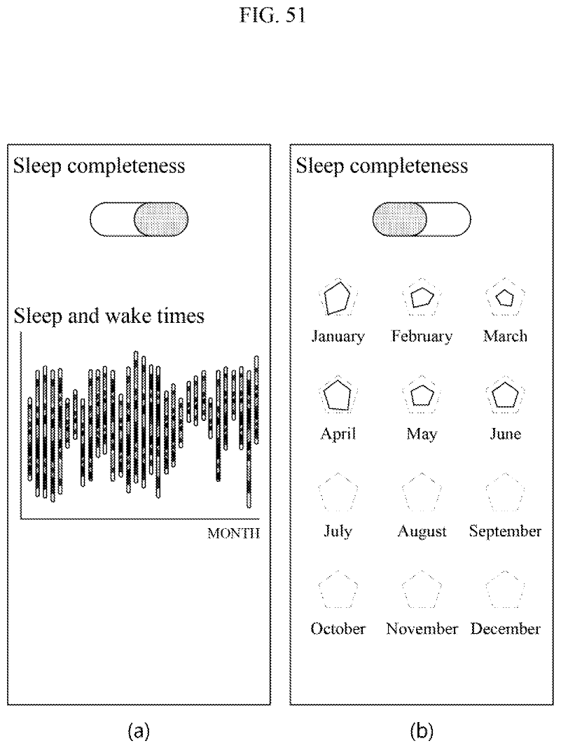

As illustrated in (a) of FIG. 51, the user terminal 3000 may display the phase of sleep of the user determined during sleep on a timeline through a display and provide it to the user on a monthly basis or yearly basis, and, as illustrated in (b) of FIG. 51, may provide an object generated based on at least one of the first sleep score to the fifth sleep score to the user on a monthly basis or yearly basis through a display.

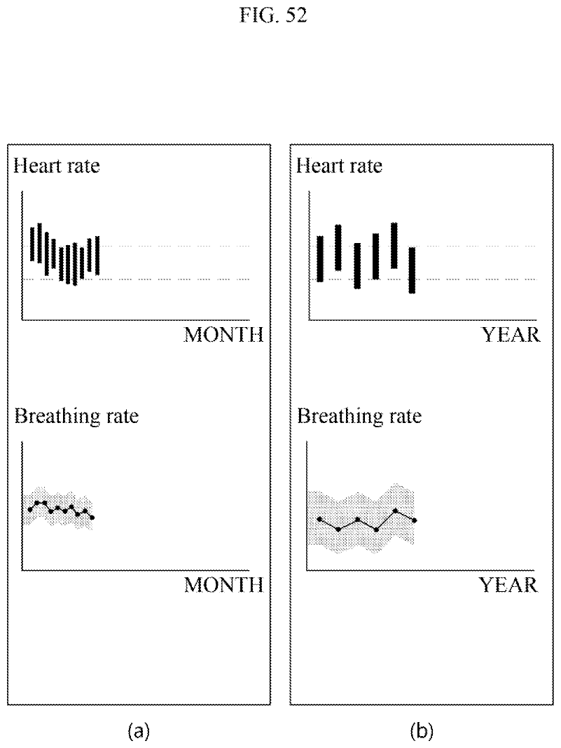

The user terminal 3000 may visually display and output indicators that are criteria for deciding the first sleep score to the fifth sleep score through a display. For example, the user terminal 3000 may display and output a first indicator (e.g., heart rate) and a second indicator (e.g., breathing rate) extracted from at least one bio-signal on a monthly basis as illustrated in (a) of FIG. 52. As illustrated in (b) of FIG. 52, the user terminal 3000 may display and output the first indicator (e.g., heart rate) and the second indicator (e.g., breathing rate) extracted from at least one bio-signal on a yearly basis.

The features, structures, effects, etc. described in the embodiments above are included in at least one embodiment of the present invention, and are not necessarily limited to only one embodiment. Furthermore, the features, structures, effects, etc. illustrated in each embodiment can be implemented by being combined or modified with respect to other embodiments by those skilled in the art in the field to which the embodiments belong. Therefore, contents related to these combinations and modifications should be construed as being included in the scope of the present invention.

In addition, although the description has been made focusing on the embodiment, this is only an example and does not limit the present invention. Those skilled in the art to which the present invention belongs will understand that various modifications and applications not exemplified above are possible within a range that does not deviate from the essential characteristics of the present embodiment. That is, each of the components specifically illustrated in the embodiment can be modified and implemented. Also, differences related to these modifications and applications should be construed as being included in the scope of the present invention as defined in the appended claims.

1. A device for predicting a sleep stage comprising:
a memory; and
at least one processor;
wherein the at least one processor is configured to:
obtain a first bio-signal and a second bio-signal measured during sleep of the user;
generate an expected sleep stage using a first neural network model based on the first bio-signal wherein the expected sleep stage comprises a first sleep stage, a second sleep stage and a third sleep stage, and
wherein the expected sleep stage is determined to be the first sleep stage if the value obtained from the first neural network satisfies a first condition, and
wherein the expected sleep stage is determined to be the second sleep stage if the value obtained from the first neural network satisfies a second condition, and
wherein the expected sleep stage is determined to be the third sleep stage if the value obtained from the first neural network satisfies a third condition;
detect a non-sleep stage of the user based on the second bio-signal;
detect an apnea using a third neural network model based on the first bio-signal and the second bio-signal; and
generate a corrected sleep stage based on the result of detecting the non-sleep stage and the result of detecting the apnea;
wherein the first bio-signal is a ballistocardiogram signal,
wherein the second bio-signal is a sound signal,
wherein the corrected sleep stage is a calibration of the expected sleep stage using the result of detecting the non-sleep stage and the result of detecting the apnea as indicators, and
wherein the result of detecting the apnea is determined based on a breathing rate derived from the first bio-signal and a sleep sound derived from the second bio-signal,
wherein the at least one processor is further configured to determine a timing for providing a wake-up alarm based on the corrected sleep stage.

2. The device of claim 1,
wherein the at least one processor is configured to extract at least one indicator based on the second bio-signal, and detect the non-sleep stage based on the at least one indicator.

3. The device of claim 2,
wherein the at least one indicator is at least one of the movement of the user, presence and absence of the user, the movement of an eye of the user, an activity amount, and entropy.

4. The device of claim 1,
wherein the at least one processor is configured to:
generate the expected sleep stage by determining, based on the first bio-signal, that the user is in the first sleep stage in a first time period, the second sleep stage in a second time period, and the third sleep stage in a third time period, and
generate the corrected sleep stage by updating the expected sleep stage by determining that the user is the non-sleep stage in the second time period, if the user is determined to be in the non-sleep stage in the second time period based on the second bio-signal.

5. The device of claim 4,
wherein the first time period, the second time period and the third time period are time periods having different lengths,
wherein the at least one processor is configured to:
determine the first sleep stage, the second sleep stage and the third sleep stage based on the first bio-signal measured during the first time period, the second time period and the third time period using the first neural network model.

6. The device of claim 4,
wherein the first time period, the second time period and the third time period are time periods having different lengths,
wherein the first neural network model comprises a first part, a second part and a third part, and
wherein the at least one processor is configured to:
determine the first sleep stage based on the first bio-signal measured during the first time period using the first part of the first neural network model,
determine the second sleep stage based on the first bio-signal measured during the second time period using the second part of the first neural network model, and
determine the third sleep stage based on the first bio-signal measured during the third time period using the third part of the first neural network model.

7. The device of claim 1,
wherein the at least one processor is configured to generate a final sleep stage using a second neural network model, and
wherein the second neural network model generates the final sleep stage using the result of detecting the non-sleep stage or the corrected sleep stage as input data.

8. The device of claim 7,
wherein the at least one processor is configured to:
obtain a third bio-signal measured during sleep of the user; and
generate the final sleep stage using the second neural network model,
wherein the second neural network model generates the final sleep stage using at least one of the result of detecting the non-sleep stage, the corrected sleep stage and the third bio-signal as input data, and
wherein the third bio-signal a ballistocardiogram signal.

9. The device of claim 8,
wherein the at least one processor is configured to:
detect the apnea using a third neural network model based on the third bio-signal; and
generate the final sleep stage using the second neural network model,
wherein the second neural network model generates the final sleep stage using at least one of the result of detecting the non-sleep stage, the result of detecting the apnea and the corrected sleep stage.

10. The device of claim 9,
wherein the at least one processor is configured to:
extract a first indicator related to breathing rate and a second indicator related to breathing amplitude based on the third bio-signal, and
detect the apnea using the third neural network model based on the first indicator and the second indicator.

11. The device of claim 9,
wherein the at least one processor is configured to:
extract the number of occurrences of apnea during the predetermined time period based on the third bio-signal using the third neural network model, and
generate the final sleep stage based on the number of occurrences of apnea.

12. The device of claim 1,
wherein the at least one processor is configured to:
obtain a third bio-signal measured during sleep of the user; and
detect the apnea using a third neural network model based on the third bio-signal.

13. A method of predicting a sleep stage, comprising:
obtaining a first bio-signal and a second bio-signal measured during sleep of a user;
generating an expected sleep stage using a first neural network model based on the first bio-signal, wherein the expected sleep stage comprises a first sleep stage, a second sleep stage and a third sleep stage, and
wherein the expected sleep stage is determined to be the first sleep stage if the value obtained from the first neural network satisfies a first condition, and
wherein the expected sleep stage is determined to be the second sleep stage if the value obtained from the first neural network satisfies a second condition, and
wherein the expected sleep stage is determined to be the third sleep stage if the value obtained from the first neural network satisfies a third condition;
detecting a non-sleep stage of the user based on the second bio-signal;
detecting an apnea using a third neural network model based on the first bio-signal and the second bio-signal; and
generating a corrected sleep stage based on the result of detecting the non-sleep stage and the result of detecting the apnea;
wherein the first bio-signal is a ballistocardiogram signal,
wherein the second bio-signal is a sound signal,
wherein the corrected sleep stage is a calibration of the expected sleep stage using the result of detecting the non-sleep stage and the result of detecting the apnea as an indicator, and
wherein the result of detecting of the apnea is determined based on a breathing rate determined based on the first bio-signal and a sleep sound determined based on the second bio-signal,
wherein the at least one processor is further configured to provide a wake-up alarm based on the corrected sleep stage.

14. A non-transitory computer readable recording medium including a program for executing a control method of an electronic device, wherein the control method comprises:
obtaining a first bio-signal and a second bio-signal measured during sleep of a user;
generating an expected sleep stage using a first neural network model based on the first bio-signal—wherein the expected sleep stage comprises a first sleep stage, a second sleep stage and a third sleep stage, and
wherein the expected sleep stage is determined to be the first sleep stage if the value obtained from the first neural network satisfies a first condition, and
wherein the expected sleep stage is determined to be the second sleep stage if the value obtained from the first neural network satisfies a second condition, and
wherein the expected sleep stage is determined to be the third sleep stage if the value obtained from the first neural network satisfies a third condition;
detecting a non-sleep stage of the user based on the second bio-signal;
detecting an apnea using a third neural network model based on the first bio-signal and the second bio-signal; and
generating a corrected sleep stage based on the result of detecting the non-sleep stage and the result of detecting the apnea;
wherein the first bio-signal is a ballistocardiogram signal,
wherein the second bio-signal is a sound signal,
wherein the corrected sleep stage is a calibration of the expected sleep stage using the result of detecting the non-sleep stage and the result of detecting the apnea as an indicator, and
wherein the result of detecting of the apnea is determined based on a breathing rate determined based on the first bio-signal and a sleep sound determined based on the second bio-signal,
wherein the at least one processor is further configured to provide a wake-up alarm based on the corrected sleep stage.
